(19) United States
(12) Patent Application Publication
(10) Pub. No.: US 2013/0238091 A1
(43) Pub. Date: Sep. 12, 2013

(54) MASKED OCULAR DEVICE FOR IMPLANTATION ADJACENT TO AN INTRAOCULAR LENS

(71) Applicant: ACUFOCUS, INC., Irvine, CA (US)

(72) Inventors: Randall Danta, Tustin, CA (US); Bruce Christie, Claremont, CA (US); Robert Grant, Laguna Beach, CA (US)

(73) Assignee: ACUFOCUS, INC., Irvine, CA (US)

(21) Appl. No.: 13/767,786

(22) Filed: Feb. 14, 2013

Related U.S. Application Data
(60) Provisional application No. 61/599,850, filed on Feb. 16, 2012.

(51) Int. Cl. A61F 2/16

(52) U.S. Cl. CPC A61F 2/1613
USPC 623/6.43

(57) ABSTRACT

The present application describes a device and methods that use a small-aperture mask surgically implanted in the optical path to improve the depth of focus of, for example, a pseudophakic patient. The device can be inserted adjacent to an intraocular lens (IOL). The device may include one or more connectors for attaching the device to an intraocular lens.

### CROSS-REFERENCE TO RELATED APPLICATIONS

This application claims a priority benefit under 35 U.S.C. §119(e) to U.S. Provisional Application No. 61/599,850, filed Feb. 16, 2012, entitled “MASKED OCULAR DEVICE FOR IMPLANTATION ADJACENT TO AN INTRAOCULAR LENS,” which is hereby incorporated by reference in its entirety.

### BACKGROUND

1. Field

This application relates generally to the field of intraocular devices. More particularly, this application is directed to small-aperture ocular devices that can be applied adjacent to an intraocular lens (IOL) and the surgical methods for implanting the ocular devices.

2. Description of the Related Art

Intraocular lenses (IOLs) are well known as a safe and effective means to treat aphakia following the removal of a cataract. IOLs are available in a variety of optical and mechanical designs, incorporating features that provide a fixed focus (conventional or “monofocal” lenses), multiple fixed foci (“multifocal” lenses), or variable focus (“accommodating” lenses). While some degree of success has been achieved with multifocal and accommodating lenses, they require compromises on the part of the patient or surgeon that many find objectionable.

Monofocal IOLs provide excellent vision at near or far, but not both. Patients with monofocal IOLs typically use spectacles (e.g., reading glasses) for the distances where they are not well focused. Multifocal IOLs provide the simultaneous ability to see near and far, but they also introduce contrast losses and symptoms, particularly at night. Accommodating IOLs are a more recent development showing promise, but so far, these lenses are large, complicated, and bear higher surgical risks than the alternatives. They also face a challenge in properly restoring accommodative function in all patients because of variations in the size and properties of the eye structures that participate in the accommodative action.

The inability to focus at different distances usually begins after age 40, but the onset of cataracts is much later, usually after age 65. Thus, a large number of patients between about age 40 and about age 65 often retain excellent distance vision, but can no longer read up close; a condition known as presbyopia. Vorosmarthy and Miller have taught how placing a small aperture in an intraocular lens can improve depth of focus, providing patients focused vision at distance with sufficient depth of field to read up close.

### SUMMARY

Certain aspects of this disclosure are directed toward an intraocular device including a mask. The intraocular device can include one or more of the features of the intraocular devices described herein. The mask can be configured to increase the depth of focus of a patient. The mask can include an aperture configured to transmit substantially all visible light. The mask can include a non-transmissive region surrounding the aperture. The non-transmissive region can be configured to be substantially opaque to visible light. The mask can include one or more connectors configured to attach the mask to an intraocular lens.

In the above mentioned intraocular device, the one or more of the connectors can include one or more spring clips.

In any of the above mentioned intraocular devices, one or more of the connectors can include one or more hooks. In certain aspects, at least a portion of each hook can include a curvature generally consistent with a curvature of the mask.

In any of the above mentioned intraocular devices, the one or more connectors can be configured to attach to one or more haptics of the intraocular lens.

In any of the above mentioned intraocular devices, the one or more connectors can be configured to engage an outer periphery of the intraocular lens.

In any of the above mentioned intraocular devices, the one or more connectors can be positioned at an outer periphery of the mask.

In any of the above mentioned intraocular devices, the one or more connectors can include at least two connectors spaced around an outer periphery of the mask.

In any of the above mentioned intraocular devices, the mask can include a plurality of holes disposed in the non-transmissive region, the plurality of holes positioned at irregular locations to reduce visible diffraction patterns due to the transmission of visible light through the holes.

In any of the above mentioned intraocular devices, at least a portion of the non-transmissive region can include a texturized surface. In certain aspects, the portion of the non-transmissive region can be at least about 50% of the non-transmissive region. In certain aspects, the texturized surface can include a surface roughness of less than about 125 microinches.

In any of the above mentioned intraocular devices, the mask can include a substantially transparent outer region surrounding at least a portion of the non-transmissive region.

In any of the above mentioned intraocular devices, the mask can include nanites configured to selectively transmit light.

In any of the above mentioned intraocular devices, the one or more connectors can be configured to removably attach the mask to the intraocular lens.

In any of the above mentioned intraocular devices, the mask can include a curvature.

In any of the above mentioned intraocular devices, the curvature of the mask can generally match the curvature of the intraocular lens.

In any of the above mentioned intraocular devices, the mask can include at least one haptic configured to support the mask within the eye of a patient.

In any of the above mentioned intraocular devices, the aperture of the mask can include a diameter of about 1.2 mm to about 2.0 mm.

In any of the above mentioned intraocular devices, the mask can include an outer diameter between about 3.2 mm and 3.8 mm.

In any of the above mentioned intraocular devices, the intraocular device can also include an intraocular lens connected to the mask by the one or more connectors.

In any of the above mentioned intraocular devices, the aperture can include a generally circular or generally oval shape. The aperture can include other shapes, examples of which are described in U.S. Pat. No. 7,628,810, issued Dec. 8, 2009, which is hereby incorporated by reference in its entirety.

Certain aspects of this disclosure are directed toward a method of implanting an intraocular device. The method can include one or more of the method steps described herein. In certain aspects, the method can include creating a surgical incision in an eye. In certain aspects, the method can include implanting an intraocular lens in an intraocular space. In certain aspects, the method can include implanting an intraocular device adjacent to the intraocular lens. In certain aspects, the intraocular device can include a mask configured to increase the depth of focus of a patient. The mask can include an aperture configured to transmit substantially all visible light and a non-transmissive region surrounding the aperture. The non-transmissive region can be configured to be substantially opaque to visible light.

In the above mentioned method aspect, implanting the intraocular lens can occur during a prior procedure completed before creating the surgical incision in the eye.

In any of the above mentioned method aspects, implanting the intraocular lens can occur before implanting the intraocular device.

In any of the above mentioned method aspects, the method can include removing the intraocular device. In certain aspects, the method can include removing the intraocular device and maintaining the position of the intraocular lens within the eye.

In any of the above mentioned method aspects, the method can include attaching the intraocular device to the intraocular lens. In certain aspects, attaching the intraocular device to the intraocular lens can occur before implanting the intraocular lens. In certain aspects, attaching the intraocular device to the intraocular lens can include attaching one or more intraocular device hooks to the intraocular lens. In certain aspects, attaching the intraocular device to the intraocular lens can include attaching one or more intraocular device clips to the intraocular lens. In certain aspects, attaching the intraocular device to the intraocular lens can include attaching the intraocular device to an outer periphery of the intraocular lens. In certain aspects, attaching the intraocular device to the intraocular lens can include attaching the intraocular device to one or more haptics of the intraocular lens.

In any of the above mentioned method aspects, implanting the intraocular device can include implanting the intraocular device in an anterior chamber of the eye.

In any of the above mentioned method aspects, implanting the intraocular device can include implanting the intraocular device within a lens capsule.

In any of the above mentioned method aspects, implanting the intraocular device can include implanting the intraocular device into a sulcus-region of the eye.

Certain aspects of this disclosure are directed toward an intraocular device configured to attach to an anterior surface of a lens capsule of a patient. The intraocular device can include any of the intraocular device features described herein. The intraocular device can include a mask. The mask can include a transmissive region configured to transmit light and a non-transmissive region configured to block at least some visible light incident thereon. The non-transmissive region can surround at least a portion of the transmissive region. The mask can include one or more protrusions positioned near an outer periphery of the mask. The one or more protrusions can be configured to attach to the anterior surface of the lens capsule of a patient.

In any of the above mentioned intraocular devices, the mask can include one or more protrusions positioned at the outer periphery of the mask.

In any of the above mentioned intraocular devices, the mask can include one or more protrusions extending generally outwardly from an anterior surface of the mask.

In any of the above mentioned intraocular devices, the mask can include one or more protrusions integrally formed with the mask.

In any of the above mentioned intraocular devices, the mask can include one or more protrusions and each protrusion can include a curvature.

In any of the above mentioned intraocular devices, the mask can include one or more protrusions surrounding substantially the entire outer periphery of the mask.

Certain aspects of this disclosure are directed toward a method of implanting an intraocular device. The method can include any of the method steps described herein. The method can include creating a surgical incision in an eye to access an intraocular space. The method can include performing a capsulotomy procedure. The method can include mounting an intraocular device to a lens capsule of the eye. The intraocular device can include a mask configured to increase the depth of focus of a patient. The mask can include one or more protrusions near an outer periphery of the mask for mounting the mask to the lens capsule. In certain aspects, mounting the intraocular device to the lens capsule can include positioning the intraocular device within a capsulotomy incision on the lens capsule.

Certain aspects of this disclosure are directed to a small-aperture ocular device that allows patients to focus at a distance with sufficient depth of field to read up close. The ocular device of the present application is significantly improved over previous small-aperture devices at least because the ocular device can be an independent implant that can be inserted adjacent to any IOL that the surgeon prefers or any IOL required by the patient's particular clinical characteristics. For example, the ocular device can be inserted adjacent to monofocal IOLs, multifocal IOLs, accommodating IOLs, and toric IOLs. The ocular device can be implanted in a variety of locations along the optical pathway in the eye, including adjacent to the anterior surface of an IOL, the posterior surface of an IOL, adjacent to or within the capsular bag, or between the iris and cornea.

The ocular device can include a mask having a substantially annular non-transmissive region surrounding a relatively high transmissive central region, such as a clear lens or aperture. The device can have an annular mask with a small aperture for light to pass through to the retina to increase depth of focus, sometimes referred to herein as pinhole effect, pinhole imaging or pinhole vision correction. The ocular device can include at least one retention member, such as at least one haptic for example, to support the ocular device after implantation into an eye.

The ocular device can be implanted into an eye through a variety of methods. For example, the device can be joined to an IOL prior to implantation, and the device and IOL can be implanted simultaneously. In certain aspects, the device and an IOL can be implanted sequentially, although the implantation can occur through one incision during a single procedure. In certain aspects, the device can be implanted adjacent to a patient's previously implanted IOL.

Any of the ocular devices described herein can include a mask to increase the depth of focus of the patient. The mask can have a first and second surface, wherein one of the first or second surfaces can be shaped and configured to be placed adjacent to an IOL. One region of the mask can be non-transmissive, or substantially opaque to incident light. A central aperture of the mask can be transparent to substantially all light in the visible range.

The mask can include any of the features described herein. For example, one surface of the device can have a concave shape that substantially corresponds to a convex surface of an IOL. In certain aspects, the mask can include a small gap between at least some parts of the second surface of the device and the surface of the IOL. In certain aspects, one surface of the device can include a relatively planar or flat shape, such that when placed adjacent to an IOL a circle of contact can be formed between the second surface of the device and a surface of the IOL. In certain aspects, the non-transmissive region of the device can be substantially opaque to visible light, while remaining at least partially transparent to infrared (IR) light. Examples of devices at least partially transparent to IR light can be found in U.S. application Ser. No. 13/691,625, filed Nov. 30, 2012, titled “Ocular Mask Having Selective Spectral Transmission,” which is hereby incorporated by reference in its entirety.

The ocular devices disclosed in this specification can include any of the retention members described herein. In certain aspects, the device can comprise at least one retention member configured to support the device after implantation into an eye of a patient. The at least one retention member can substantially maintain centration of the central aperture along an optical axis of an eye, in some embodiments. In certain aspects, the retention member can comprise at least one haptic extending generally outwardly from the mask. The at least one haptic can be a separate piece attached to the mask by a connector. In certain aspects, the at least one haptic can be integrally formed with the mask.

In certain aspects, a mask can be applied on a surface of a transparent one-piece body. The transparent body can comprise at least one retention member, such as at least one haptic, extending generally outwardly from the mask. The mask may be printed, bonded, adhered, etched, or mechanically attached to the body, or may be embedded within the body. The mask can be applied in a generally annular shape so that a central, un-masked portion remains. The central un-masked portion can comprise material of the transparent body or an opening with no material in the transparent body.

In certain aspects, the central un-masked portion can have an optical power to correct refractive errors of the patient at the same time as providing the increased depth of field.

The ocular devices disclosed herein can be implanted in or affixed to any portion of the eye disclosed herein. The device can be configured for placement in the anterior chamber of the eye, fixating in the anterior chamber. The device can be configured to be attached to the iris using at least one haptic with at least one claw. The device can be configured for placement in the posterior chamber of the eye, wherein the device can be attached to the ciliary sulcus or fixated in the capsular bag. An ocular device with grooved edges or protrusions on or near the outer periphery of the device can be provided. The grooved edges can be configured such that the device can be attached to the anterior surface of a lens capsule. Hooks can be provided on or near the outer periphery of a mask. The hooks can be configured such that the mask can be attached to any of a variety of IOLs. The hooks can be configured so as to allow implantation of the mask adjacent to a previously implanted IOL.

The ocular devices disclosed herein can be implanted using any of the methods described herein. The device can be implanted through a small incision after being joined to an IOL. The device and IOL can be permanently joined. The device and IOL can be temporarily joined. An ocular device can be implanted sequentially with an IOL through one incision and during a single procedure. An ocular device can be implanted adjacent to an existing IOL that was implanted in a previous procedure.

For purposes of summarizing the disclosure, certain aspects, advantages and features of the inventions have been described herein. It is to be understood that not necessarily any or all such advantages will be achieved in accordance with any or all particular embodiments of the inventions disclosed herein.

### BRIEF DESCRIPTION OF THE DRAWINGS

Certain features, aspects, and advantages of the subject matter disclosed herein are described below with reference to the drawings, which are intended to illustrate and not to limit the scope of the disclosure. Various features of different disclosed embodiments can be combined to form additional embodiments, which are part of this disclosure. No structures, features, steps, or processes are essential or critical; any can be omitted in certain embodiments. The drawings comprise the following figures.

FIG. 1A illustrates a front view of one embodiment of an ocular device with a mask with connector portions for haptic retention as described herein.

FIG. 1B illustrates a side view of the embodiment of FIG. 1A.

FIG. 2A illustrates a front view of one embodiment of an ocular device with a mask with folded connector portions for haptic retention as described herein.

FIG. 2B illustrates a side view of the embodiment of FIG. 2A.

FIG. 3A illustrates a front view of one embodiment of an ocular device with a mask with integral haptics as described herein.

FIG. 3B illustrates a side view of the embodiment of FIG. 3A.

FIG. 4 illustrates a front view of one embodiment of a substantially flat ocular device with a mask with integral haptics as described herein.

FIG. 5 illustrates a front view of one embodiment of an ocular device with a mask applied on a transparent body.

FIG. 6A illustrates a front view of one embodiment of an ocular device with a mask printed on a transparent body having optical power in a center portion.

FIG. 6B illustrates a side view of the embodiment of FIG. 6A.

FIG. 7A illustrates one embodiment of an ocular device with a mask with retention members for supporting the device in the anterior chamber of an eye.

FIG. 7B illustrates a side view of the embodiment of FIG. 7A.

FIG. 8A illustrates one embodiment of an ocular device with a mask with retention members for securing the device to an iris of an eye.

FIG. 8B illustrates a side view of the embodiment of FIG. 8A.

FIG. 9A illustrates a front view of one embodiment of an ocular device with a mask with hooks.

FIG. 9B illustrates a side view of the embodiment of FIG. 9A.

FIG. 10A illustrates a front view of one embodiment of an ocular device with a mask with spring clips.

FIG. 10B illustrates a side view of the embodiment of FIG. 10A.

FIG. 10C illustrates details of a spring clip of the embodiment of FIG. 10A attached to the haptic of an IOL.

FIG. 11A illustrates a front view of one embodiment of an ocular device with a mask with hooks attached to a one-piece IOL.

FIG. 11B illustrates a side view of the embodiment of FIG. 11A attached to a one-piece IOL.

FIG. 11C illustrates an oblique view of the embodiment of FIG. 11A attached to a one-piece IOL.

FIG. 12A illustrates a front view of one embodiment of an ocular device with a mask with hooks attached to a type of IOL.

FIG. 12B illustrates a side view of one embodiment of an ocular device with a mask with hooks attached to a type of IOL.

FIG. 12C illustrates another side view of one embodiment of an ocular device with a mask with hooks attached to a type of IOL.

FIG. 13A illustrates a front view of one embodiment of an ocular device with a mask with protrusions for supporting the device on a lens capsule.

FIG. 13B illustrates a side view of the embodiment of FIG. 13A.

FIG. 13C illustrates details of the embodiment of the ocular device of FIG. 13A.

FIG. 14A illustrates a front view of one embodiment of an ocular device with a mask with protrusions for supporting the device attached to a lens capsule.

FIG. 14B illustrates a side view of the embodiment of FIG. 14A attached to a lens capsule.

FIG. 14C illustrates details of the embodiment of FIG. 14A attached to a lens capsule.

FIG. 14D illustrates an oblique view of the embodiment of FIG. 14A attached to a lens capsule.

FIG. 15 illustrates an enlarged, diagrammatic view of an embodiment of a mask that includes particulate structure adapted for selectively controlling light transmission through the mask in a low light environment.

FIG. 16 illustrates the mask of FIG. 15 in a bright light environment.

### DETAILED DESCRIPTION

This application is directed to an ocular device for implantation adjacent to an IOL for improving the depth of focus of an eye of a patient and surgical methods for implanting the ocular device. The ocular device can include a mask configured to be positioned adjacent to an intraocular lens (IOL). The masks can comprise an annular shape with a small aperture to provide vision correction. The device may be applied to the eye in any of a variety of manners and in any location along the optical path. For example, the device can be implanted in the anterior chamber or the posterior chamber. As a further example, in the posterior chamber, the device can be attached to the ciliary sulcus (“sometimes referred to herein as “sulcus-fixated”). As a further example, the device can be implanted within an eye's lens capsule or on an outer surface of the lens capsule. The device can be positioned adjacent to an IOL. The ocular device can be implanted into an eye adjacent to any type of IOL, including monofocal IOLs, multifocal IOLs, and accommodating IOLs. A variety of techniques can be used to make the ocular device suitable for positioning adjacent an IOL, such as selecting a complementary curvature, selecting a material that is particularly compatible with eye tissue and fluids, or selecting suitable thickness or range of thicknesses. These features are discussed further below.

### I. Ocular Device

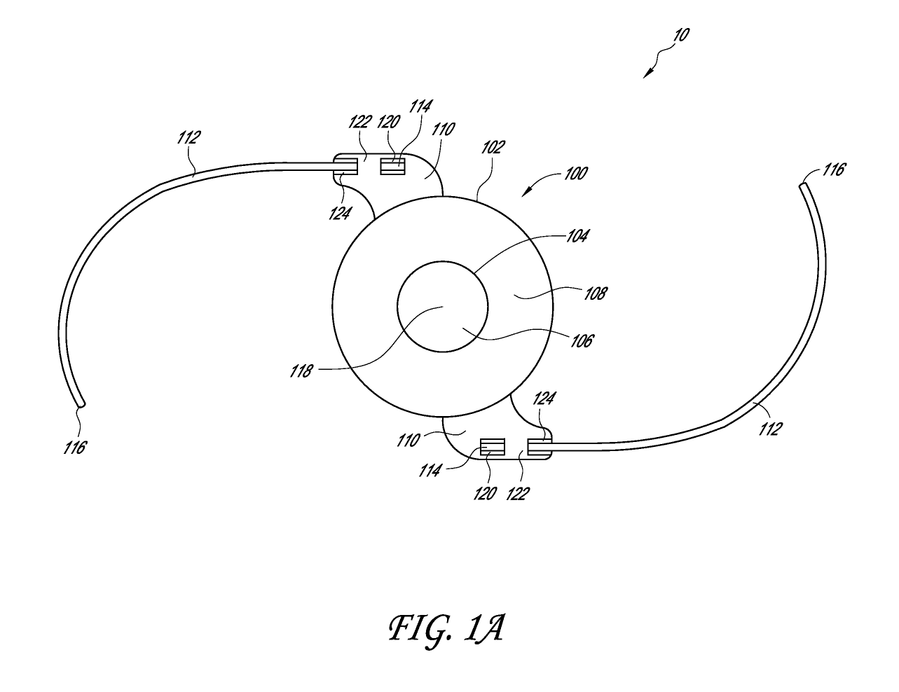

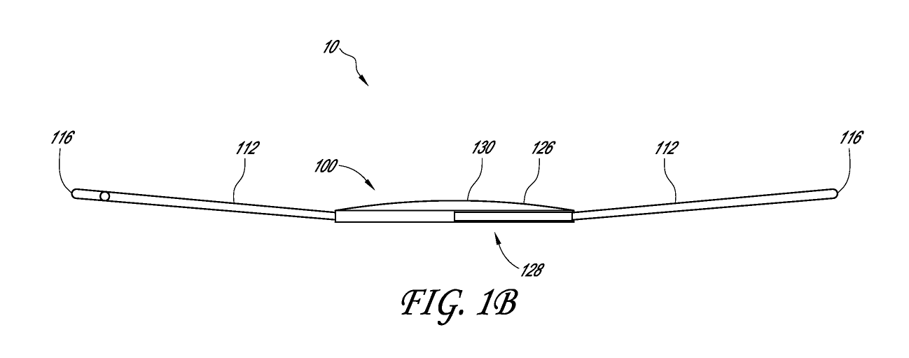

FIGS. 1-12C illustrates further details of the ocular device and various embodiments thereof. In some embodiments, the ocular device is designed to be positioned adjacent to an IOL. Various IOLs exist to suit the vision correction needs of particular patients. Generally, however, IOLs comprise a lens body with an optical power to refract light and correct refractive errors of the eye. The lens body of a typical IOL generally includes an anterior surface and a posterior surface. The anterior surface and posterior surface can each comprise preselected curvatures. In many IOLs, both the anterior and posterior surfaces of the IOL are convex.

As shown in FIGS. 1 and 1B, in one embodiment, the ocular device 10 can comprise a mask 100, wherein the mask 100 includes an annular region 108 surrounding a small aperture 106 substantially centrally located within the mask 100. The aperture can be generally located around a central axis 130 of the mask. The mask can be generally symmetrical about the central axis 130. However, in some embodiments, masks that are asymmetrical may provide benefits, such as enabling a mask to be located or maintained in a selected position with respect to the anatomy of the eye.

In certain embodiments, the mask 100 can have an inner periphery 104 and an outer periphery 102. The outer periphery 102 can take any suitable form. In some embodiments, the outer periphery 102 can be generally circular, being defined by an outer circumference of the ocular device. In some embodiments, the circumference defining the outer periphery 102 can be at least about 8 mm and/or less than or equal to about 30 mm. In some embodiments, the circumference defining the outer periphery 102 can be at least about 10 mm and/or less than or equal to about 20 mm. The inner periphery 104 can also take any suitable form. In some embodiments, the inner periphery 104 can be generally circular, being defined by a circumference of a small inner aperture 106 of the mask 100. In some embodiments, the circumference defining the inner periphery 104 can be greater than zero and/or less than or equal to about 8 mm. In some embodiments, the circumference defining the inner periphery 104 can be between at least about 1 mm and/or less than or equal to about 4.5 mm, or in some embodiments the circumference of the inner periphery 104 can be at least about 3 mm and/or less than or equal to about 6 mm.

In some embodiments, the mask 100 can have a diameter of at least about 3 mm and/or less than or equal to about 8 mm, often within the range of from about 3.5 mm to about 6 mm. In some embodiments, the mask 100 can be substantially circular and can include a diameter of at least 3.5 mm and/or less than or equal to 4 mm. In some embodiments, the mask 100 can be substantially circular and can include a diameter of less than 4 mm. The outer periphery 102 of the mask 100 can have a diameter of about 3.8 mm in some embodiments.

The mask 100 can have dimensions that allow the mask to be inserted into the patient's eye and improve the patient's vision. For example, the thickness of the mask 100 can vary depending on the intended location of the mask 100 within the eye. In certain embodiments, the mask 100 can have a thickness of at least about 0.001 mm and/or less than or equal to about 0.5 mm. In some embodiments, the mask 100 has a thickness of less than about 0.25 mm. In some embodiment, the mask 100 has a thickness of at least about 0.01 mm and/or less than or equal to about 0.02 mm, or from about 0.001 mm to about 0.01 mm. In some embodiments, the mask 100 can have a thickness of less than about 0.001 mm.

The mask 100 can have a constant thickness, as discussed below. However, in some embodiments, the thickness of the mask 100 may vary between an inner periphery 104 and an outer periphery 102. For example, the mask 100 can have a gradually decreasing thickness from the inner periphery 104 to the outer periphery 102. In another example, the mask 100 can have a gradually increasing thickness from the inner periphery 104 to the outer periphery 102. Other cross-sectional profiles of the mask 100 are also possible.

In certain embodiments, the mask 100 can have a first surface 126 extending between the inner periphery 104 and the outer periphery 102. The mask 100 can also have a second surface 128 extending between the inner periphery 104 and outer periphery 102. As discussed further below, the mask's 100 surfaces can be shaped and configured to reside near or conform to adjacent lens surfaces.

In some embodiments, the first surface 126 of the mask 100 can have a generally convex shape, as illustrated in FIG. 1B. In some embodiments, the second surface 128 of the mask 100 can have a concave shape. In some embodiments, both surfaces are curved such that the first surface 126 of the mask 100 has a convex shape and the second surface 128 of the mask 100 has a concave shape. In some embodiments, the concave shape of the second surface 128 allows the mask 100 to substantially correspond to a convex curvature of an anterior or posterior surface of an IOL. For example, the concave second surface 128 of the mask 100 can correspond to the convex anterior surface of the IOL, permitting the mask 100 to be positioned between the lens and the iris. As a further example, the concave second surface 128 of the mask 100 can correspond to the convex posterior surface of the IOL, permitting the mask 100 to be positioned between the lens and the retina.

In some embodiments, the first and second surfaces of the mask 100 can be substantially planar or flat, so that very little or no uniform curvature can be measured across the planar surfaces. In one embodiment, a substantially flat mask 100 can be placed in a variety of positions in the eye, including posterior to the IOL, anterior to the IOL, or between the iris and cornea. In some embodiments the substantially flat mask 100 can also be positioned to abut the IOL, so that a portion of the mask 100 touches the IOL. In certain embodiments, the substantially flat mask 100 can touch the IOL so that a substantially circle-shaped contact area between the mask and the IOL is created. A substantially flat mask can have several advantages over a non-planar mask. For example, a substantially planar mask can be fabricated more easily than one that has to be made to a particular curvature. In particular, the process steps involved in inducing curvature in the mask 100 can be eliminated.

In some embodiments, the first and second surfaces of the mask 100 can both be substantially circular in shape from a frontal view. In certain embodiments, the mask comprises a substantially annular non-transmissive region surrounding a relatively high transmissive central region, such as a clear lens or aperture, discussed further below. In some embodiments, the mask can also have a substantially transparent outer region surrounding the non-transmissive region.

Any of the implants described herein can be formed, or be treated, or be coated, to minimize foreign body response, such as that associated with posterior capsule opacification (PCO). In general, PCO can be minimized or avoided by providing the implant (the mask, the IOL or both, corneal inlay or any other implant intended for implantation within the optical path) with a texturized surface. The texturized surface may be provided over at least about 50%, in some implementations at least about 75% and often at least about 85% or 90% or more of at least one surface of the implant. The texturization can be omitted from the surface of the implant at the point of the intended intersection with the optical axis.

The texture can be measured on a microinch scale. The texturization can include an average surface roughness of at least about 8 microinches and/or less than or equal to about a 125 microinch surface roughness average may be used. In some implementations, texturization may be less than about 8 microinches, less than about 16 microinches, less than about 32 microinches, or less than about 64 microinches. Texturization in the range of from at least about 8 microinches, often at least about 16 microinches and in some implementations at least about 32 microinches may be formed or applied, depending upon the desired clinical performance. In some implementations, texturization in the range of from at least about 16 microinches and/or less than or equal to about 64 microinches may be formed or applied.

Texturization may be achieved in any of a variety of ways, such as by chemical treatment, application of energy by an energy source, polishing, plasma etching, die casting, sand blasting, forming the device using a textured mold, or other mechanical treatment such as imprinting the device with a textured plate or other suitable imprinting tool.

### II. Materials for the Ocular Device

The mask portion can be formed of any suitable material, including at least one of an open cell foam material, an expanded solid material, and a substantially opaque material. In some embodiments, the material used to form the mask has relatively high water content. In some embodiments, the materials that can be used to form the mask include any of a variety of polymers (e.g., PMMA, PVDF, polypropylene, polycarbonate, PEEK, polyethylene, acrylic copolymers (e.g., hydrophobic or hydrophilic), polystyrene, PVC, polysulfone), hydrogels, silicone, metals, metal alloys, or carbon (e.g., graphene, pure carbon) and those materials further discussed below. Fibrous materials such as a Dacron mesh can also be used. The material should be biocompatible.

Because the mask has a very high surface to volume ratio and is exposed to a great deal of sunlight following implantation, the mask preferably comprises a material which has a good resistance to degradation, including from exposure to ultraviolet (UV) or other wavelengths of light. Polymers including a UV absorbing component, including those comprising UV absorbing additives or made with UV absorbing monomers (including co-monomers), may be used in forming masks as disclosed herein which are resistant to degradation by UV radiation. Examples of such polymers include, but are not limited to, those described in U.S. Pat. Nos. 4,985,559 and 4,528,311, the disclosures of which are hereby incorporated by reference in their entireties. In some embodiments, the mask comprises a material which itself is resistant to degradation by UV radiation. In one embodiment, the mask comprises a polymeric material which is substantially reflective of or transparent to UV radiation. The lens body may include a UV absorbing component in addition to the mask being resistant to degradation by UV radiation or the mask may not be resistant to degradation by UV radiation since the UV absorbing component in the lens body may prevent degradation of the mask by UV radiation.

The mask may include a component which imparts a degradation resistive effect, or may be provided with a coating, at least on the anterior surface, which imparts degradation resistance. Such components may be included, for example, by blending one or more degradation resistant polymers with one or more other polymers. Such blends may also comprise additives which provide desirable properties, such as UV absorbing materials. In some embodiments, blends can include a total of about 1-20 wt. %, including about 1-10 wt. %, 5-15 wt. %, and 10-20 wt. % of one or more degradation resistant polymers. In some embodiments, blends can include a total of about 80-100 wt. %, including about 80-90 wt. %, 85-95 wt. %, and 90-100 wt. % of one or more degradation resistant polymers. In some embodiments, the blend has more equivalent proportions of materials, comprising a total of about 40-60 wt. %, including about 50-60 wt. %, and 40-50 wt. % of one or more degradation resistant polymers. Masks may also include blends of different types of degradation resistant polymers, including those blends comprising one or more generally UV transparent or reflective polymers with one or more polymers incorporating UV absorption additives or monomers. These blends include those having a total of about 1-20 wt. %, including about 1-10 wt. %, 5-15 wt. %, and 10-20 wt. % of one or more generally UV transparent polymers, a total of about 80-100 wt. %, including about 80-90 wt. %, 85-95 wt. %, and 90-100 wt. % of one or more generally UV transparent polymers, and a total of about 40-60 wt. %, including about 50-60 wt. %, and 40-50 wt. % of one or more generally UV transparent polymers. The polymer or polymer blend may be mixed with other materials as discussed below, including, but not limited to, opacification agents, polyanionic compounds and/or wound healing modulator compounds. When mixed with these other materials, the amount of polymer or polymer blend in the material which makes up the mask can be about 50%-99% by weight, including about 60%-90% by weight, about 65-85% by weight, about 70-80% by weight, and about 90-99% by weight.

Degradation resistant polymers can include halogenated polymers, such as fluorinated polymers, that is, polymers having at least one carbon-fluorine bond, including highly fluorinated polymers as described in U.S. Pat. No. 7,976,577, issued Jul. 12, 2011, which is incorporated by reference herein in its entirety. The term “highly fluorinated” as it is used herein, is a broad term used in its ordinary sense, and includes polymers having at least one carbon-fluorine bond (C—F bond) where the number of C—F bonds equals or exceeds the number of carbon-hydrogen bonds (C—H bonds). Highly fluorinated materials also include perfluorinated or fully fluorinated materials, materials which include other halogen substituents such as chlorine, and materials which include oxygen- or nitrogen-containing functional groups. For polymeric materials, the number of bonds may be counted by referring to the monomer(s) or repeating units which form the polymer, and in the case of a copolymer, by the relative amounts of each monomer (on a molar basis).

Highly fluorinated polymers can include, but are not limited to, polytetrafluoroethylene (PFTE or Teflon®), polyvinylidene fluoride (PVDF or Kynar®), poly-1,1,2-trifluoroethylene, and perfluoroalkoxyethylene (PFA). Other highly fluorinated polymers include, but are not limited to, homopolymers and copolymers including one or more of the following monomer units: tetrafluoroethylene —(CF2-CF2)-; vinylidene fluoride —(CF2-CH2)-; 1,1,2-trifluoroethylene —(CF2-CHF)—; hexafluoropropene —(CF(CF3)-CF2)-; vinyl fluoride —(CH2-CHF)— (homopolymer is not “highly fluorinated”); oxygen-containing monomers such as —(O—CF2)-, —(O—CF2-CF2)-, —(O—CF(CF3)-CF2)-; chlorine-containing monomers such as —(CF2-CFCl)—. Other fluorinated polymers, such as fluorinated polyimide and fluorinated acrylates, having sufficient degrees of fluorination are also contemplated as highly fluorinated polymers for use in masks according to some embodiments. The homopolymers and copolymers described herein are available commercially and/or methods for their preparation from commercially available materials are widely published and known to those in the polymer arts.

Although highly fluorinated polymers are discussed herein, polymers having one or more carbon-fluorine bonds but not falling within the definition of “highly fluorinated” polymers as discussed above, may also be used. Such polymers include co-polymers formed from one or more of the monomers in the preceding paragraph with ethylene, vinyl fluoride or other monomer to form a polymeric material having a greater number of C—H bonds than C—F bonds. Other fluorinated polymers, such as fluorinated polyimide, may also be used. Other materials that could be used in some applications, alone or in combination with a fluorinated or a highly fluorinated polymer, are described in U.S. Pat. No. 4,985,559 and in U.S. Pat. No. 4,528,311, both of which are hereby incorporated by reference herein in their entirety.

The preceding definition of highly fluorinated is best illustrated by means of a few examples. One UV-resistant polymeric material is polyvinylidene fluoride (PVDF), having a structure represented by the formula: —(CF2-CH2)n-. Each repeating unit has two C—H bonds, and two C—F bonds. Because the number of C—F bonds equals or exceeds the number of C—H bonds, PVDF homopolymer is a “highly fluorinated” polymer. Another material is a tetrafluoroethylene/vinyl fluoride copolymer formed from these two monomers in a 2:1 molar ratio. Regardless of whether the copolymer formed is block, random or any other arrangement, from the 2:1 tetrafluoroethylene:vinyl fluoride composition one can presume a “repeating unit” comprising two tetrafluoroethylene units, each having four C—F bonds, and one vinyl fluoride unit having three C—H bonds and one C—F bond. The total bonds for two tetrafluoroethylenes and one vinyl fluoride are nine C—F bonds, and three C—H bonds. Because the number of C—F bonds equals or exceeds the number of C—H bonds, this copolymer is considered highly fluorinated.

Certain highly fluorinated polymers, such as PVDF, have one or more desirable characteristics, such as being relatively chemically inert and having a relatively high UV transparency as compared to their non-fluorinated or less highly fluorinated counterpart polymers. Although the applicant does not intend to be bound by theory, it is postulated that the electronegativity of fluorine may be responsible for many of the desirable properties of the materials having relatively large numbers of C—F bonds.

In some embodiments, at least a portion of the highly fluorinated polymer material forming the mask comprises an opacification agent which imparts a desired degree of opacity. In some embodiments, the opacification agent provides sufficient opacity to produce the depth of field improvements described herein, e.g., in combination with a transmissive aperture. In some embodiments, the opacification agent renders the material opaque. In some embodiments, the opacification agent prevents transmission of about 90 percent or more of incident light. In some embodiments, the opacification agent renders the material opaque. In some embodiments, the opacification agent prevents transmission of about 80 percent or more of incident light. Opacification agents can include, but are not limited to organic dyes and/or pigments, such as black ones, such as azo dyes, hematoxylin black, and Sudan black; inorganic dyes and/or pigments, including metal oxides such as iron oxide black and ilmenite, silicon carbide and carbon (e.g. carbon black, submicron powdered carbon). Although black materials are used in some embodiments, the agents can comprise any of a variety of different colors. The foregoing materials may be used alone or in combination with one or more other materials. The opacification agent may be applied to one or more surfaces of the mask on all or some of the surface, or it may be mixed or combined with the polymeric material (e.g. blended during the polymer melt phase). Although any of the foregoing materials may be used, carbon has been found to be especially useful in that it does not fade over time as do many organic dyes, and that it also aids the UV stability of the material by absorbing UV radiation. In one embodiment, carbon may be mixed with polyvinylidene fluoride (PVDF) or other polymer composition comprising highly fluorinated polymer such that the carbon comprises about 2% to about 20% by weight of the resulting composition, including about 10% to about 15% by weight, including about 12%, about 13%, and about 14% by weight of the resulting composition.

Some opacification agents, such as pigments, which are added to blacken, darken or opacify portions of the mask may cause the mask to absorb incident radiation to a greater degree than mask material not including such agents. Because the matrix polymer that carries or includes the pigments may be subject to degradation from the absorbed radiation, the mask, which is thin and has a high surface area making it vulnerable to environmental degradation, can be made of a material which is itself resistant to degradation such as from UV radiation, or that it be generally transparent to or non-absorbing of UV radiation. Use of a highly UV resistant and degradation resistant material, such as PVDF, which is highly transparent to UV radiation, allows for greater flexibility in choice of opacification agent because possible damage to the polymer caused by selection of a particular opacification agent is greatly reduced.

A number of variations of the foregoing embodiments of degradation resistant constructions are contemplated. In one variation, a mask is made almost exclusively of a material that is not subject to UV degradation. For example, the mask can be made of a metal, a highly fluorinated polymer, carbon (e.g., graphene, pure carbon), or another similar material. Construction of the mask with metal is discussed in more detail in U.S. Pat. No. 7,491,350, issued Feb. 17, 2009, and also in U.S. application Ser. No. 11/107,359 filed Apr. 14, 2005 and entitled “Method of Making an Ocular Implant”, both of which are incorporated herein in their entirety by reference. As used in this context, “exclusively” is a broad term that allows for the presence of some non-functional materials (e.g., impurities) and for an opacification agent, as discussed above. In some embodiments, the mask can include a combination of materials. For example, in one variation, the mask is formed primarily of any implantable material and is coated with a UV resistant material. In another variation, the mask includes one or more UV degradation inhibitors and/or one or more UV degradation resistant polymers in sufficient concentration such that the mask under normal use conditions will maintain sufficient functionality in terms of degradation to remain medically effective for at least about 5 years, at least about 10 years, and in certain implementations at least about 20 years.

In additional embodiments, a photochromic material can be used with the mask or in addition to the mask. Under bright light conditions, the photochromic material can darken, thereby creating a mask and enhancing near vision. Under dim light conditions, the photochromic material lightens, which allows more light to pass through to the retina. In certain embodiments, the photochromic material may be transparent to IR light at all times.

In embodiments where the ocular device has retention members, for example haptic portions, the retention members may also be formed of any suitable material, including those listed above. In embodiments where the ocular device has connector portions to connect retention members to the mask, the connector portions can be formed of any suitable material, including those listed above.

III. Ocular Devices with Retention Members

As illustrated in FIGS. 1A-8B, in certain embodiments, at least one retention member can be supplied for supporting the ocular device after implantation in an eye of the patient. In some embodiments the at least one retention member can maintain centration of the device around an optical axis of the eye. In some embodiments, at least one retention member is provided to prevent the ocular device from moving or rotating within the eye. In some embodiments, the at least one retention member is a haptic of appropriate shape and cross section to provide retention at a desired location within the eye. As used herein, the term “haptic” is intended to be a broad term encompassing struts, filaments, hooks, clips, and other mechanical structures that can be apposed against an inner surface of an eye and mounted to an ocular device structure to support an ocular device within the eye. As used herein, “a haptic” refers to at least one haptic.

The haptics can be a variety of shapes and sizes, depending on the location the ocular device is implanted in the eye and depending on the clinical characteristics of the patient. The haptics may be C-shaped, J-shaped, plate design, or any other design. The haptics may be of open or closed configuration and may be planar, angled, or step-vaulted. The haptics can have a width, the width being measured from an anterior surface to a posterior surface of the haptic, or in other words, the width being measured parallel to the patient's line of sight. The width can be in the range of at least about 0.10 mm and/or less than or equal to about 0.35 mm. In some embodiments, the haptics can have a width from about 0.10 mm to about 0.25 mm. In some embodiments, the haptics can have a width from about 0.25 mm to about 0.35 mm. The haptics can include a width of less than 0.25 mm. In some embodiments the haptics have a constant width between a proximal section and a distal end. In some embodiments, the width of the haptics can vary along their length. In some embodiments, the haptic portions can have a length of at least about 1.0 mm and/or less than or equal to about 4.5 mm measured from a peripheral edge of the implant body. In some embodiments, the haptics can be about 1.0 mm to 2.5 mm long. In some embodiments, the haptics can be about 2.5 mm to 4.5 mm long. In some embodiments, the haptics are greater than 3.0 mm long. The haptics can be flexible and can bend within the plane of the ocular device. In some embodiments, the haptics can bend out of the plane of the ocular device.

Where the ocular device comprises haptics, the radially outwardly directed force of the haptics is sufficient for stability of the ocular device within the eye, but is not so large as to cause irritation or pupil ovaling. The ocular device can exhibit a force response of approximately less than 0.5 mN, or approximately less than 0.3 mN, when the ocular device is compressed 1.0 mm according to industry standard test ISO/DIS 11979-3.

The haptics can be separate pieces attached to a mask, or the haptics can be formed integrally with the mask. As shown in FIGS. 1A and 1B, in some embodiments, a haptic 112 can be a separate piece attached to the mask 100 with a connector 110. In some embodiments, the connector 110 can extend generally outwardly from the outer periphery 102 of the mask 100, and the connector 110 can lie in a plane generally perpendicular to an optical axis. In some embodiments, the connector 110 can be attached to a first surface 126 of the mask 100. In some embodiments, the connector 110 can be attached to a second surface 128 of the mask 100. The connector 110 can be a separate piece joined to the mask 100, or the connector 110 can be formed as an extension of the mask 100. The connector 110 can be made from a variety of materials, including any of the materials used to form the mask 100, further discussed below. In some embodiments, particularly where the connector 110 is formed as an extension of the mask 100, the mask 100 and connector 110 are made from the same material. In some embodiments, the mask 100 and connector 110 can be made from different materials.

The connector can also be made in a variety of shapes. In one embodiment, best shown in FIG. 1, the connector 110 can comprise a recess 124 to receive a proximal portion of the haptic 114. In some embodiments, the recess can comprise side wall openings 120 through which the proximal portion 114 of the haptic 112 can be viewed. The openings 120 can also serve to reduce the mass of the connector 110 portion of the device. In certain embodiments, a retention band 122 can extend around the proximal portion 114 of the haptic 112 to help secure the haptic 112. In certain embodiments, the connector 110 can comprise multiple interleaving portions, arranged similarly to that of band 122, that pass over and under the proximal portion 114 to help secure the haptic 112. In some embodiments, the recess 124 can also be designed to create friction to secure the proximal portion 114 of the haptic 112. For example, the recess 124 can have a surface coating or etching to frictionally secure the proximal portion 114 to the mask 100. Other methods of securing the haptics 112 to the mask 100 include pressure staking, heat staking, chemical polymerization, and adhesives.

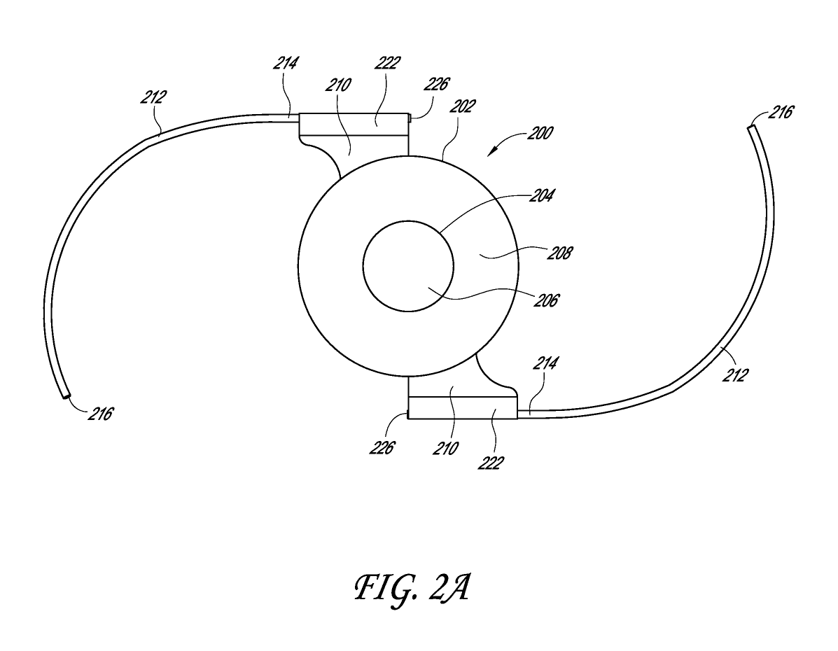

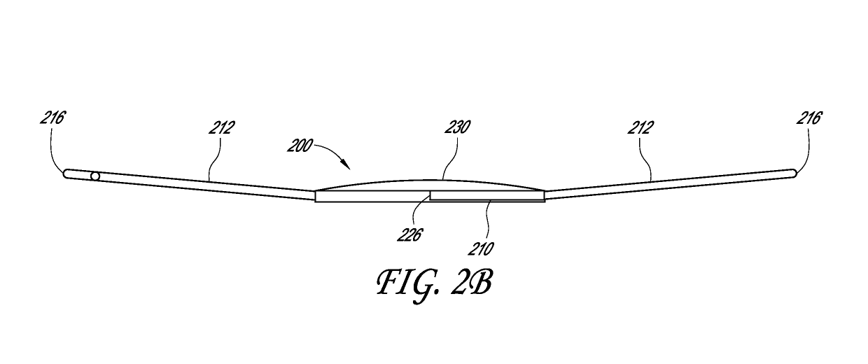

FIGS. 2A and 2B show another embodiment of an ocular device with a mask 200 with a connector 210 extending from the outer periphery 202 of the mask 200. In the illustrated embodiment, the connector 210 can have a folded region 222 that wraps around at least a portion of the proximal portion 214 of the haptic 212, thereby securing the haptic 212 to the mask 200. In certain embodiments, the proximal portion 214 can extend all the way through the folded portion of the connector 210, such that the proximal end 226 can be viewed as shown in FIG. 2.

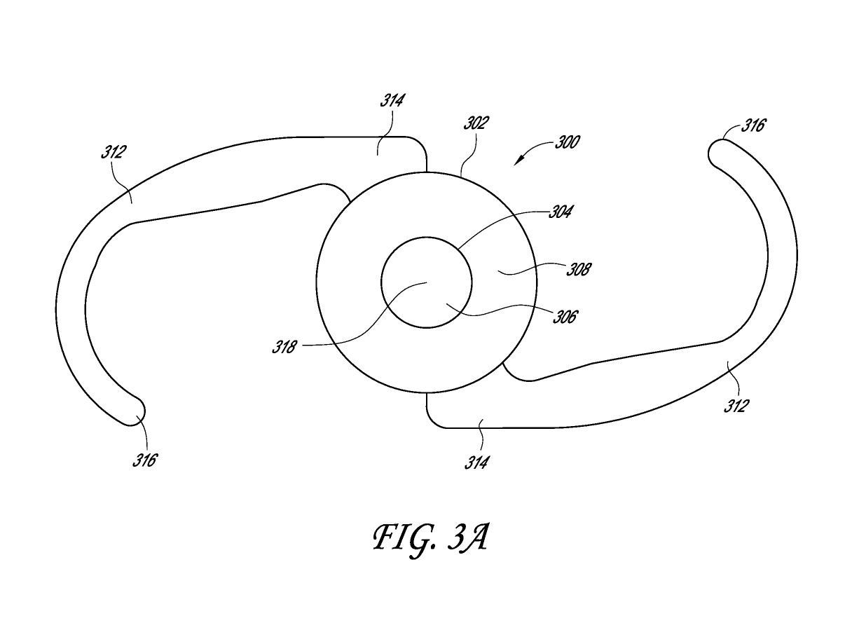

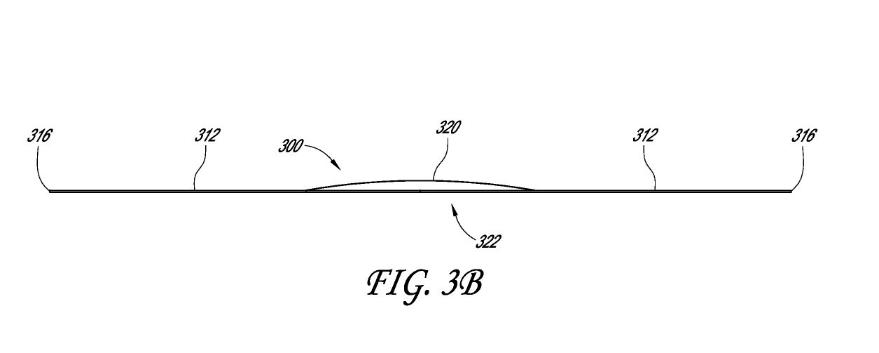

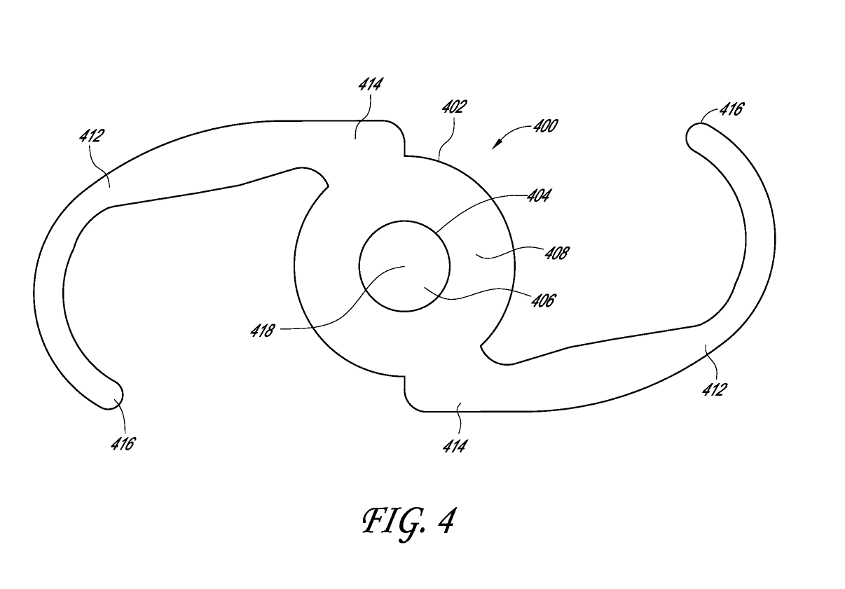

As mentioned above, haptics can also be formed integrally with a mask. FIGS. 3A-4 illustrate embodiments of an ocular device with integral haptics. In some embodiments, as shown in FIG. 4, the ocular device can be substantially planar or flat with integral haptics extending from the outer periphery 402 of the mask 400. In some embodiments, only the mask 400 portion of the device is flat and the haptics 412 can be angled or bent out of the plane of the mask in order to support the device within the eye. However, in some embodiments such as that shown in FIGS. 3A and 3B, the ocular device may not be planar or flat. In such embodiments where at least one surface of the device is curved, the haptics 312 can extend from either a first surface 320 or a second surface 322 of the mask 300, rather than extending outwardly from an outer periphery 302 of the mask 300. For example, where the first surface 320 of the mask 300 is convex and the second surface 322 of the mask 300 is concave, the haptics 312 can extend from the first surface 320 and/or from the second surface 322. In some embodiments, at least one haptic 312 can extend from the first surface 320 and at least one haptic 312 can extend from the second surface 322. In certain embodiments, an ocular device with integral haptics can be placed within a lens capsule and adjacent to an IOL. For some embodiments with integral haptics, it may be desirable for the device to have a central aperture surrounded by a mask, wherein the mask is further surrounded by a clear outer region.

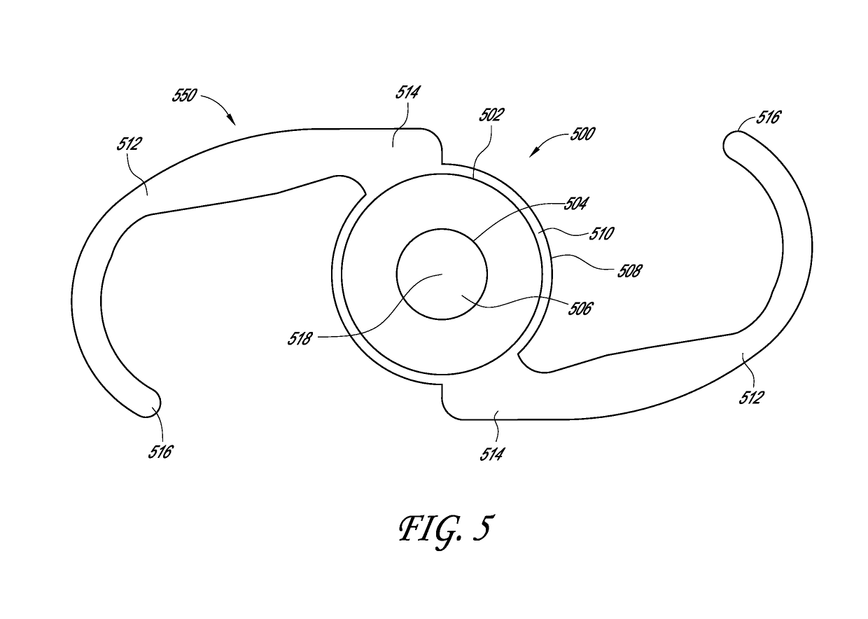

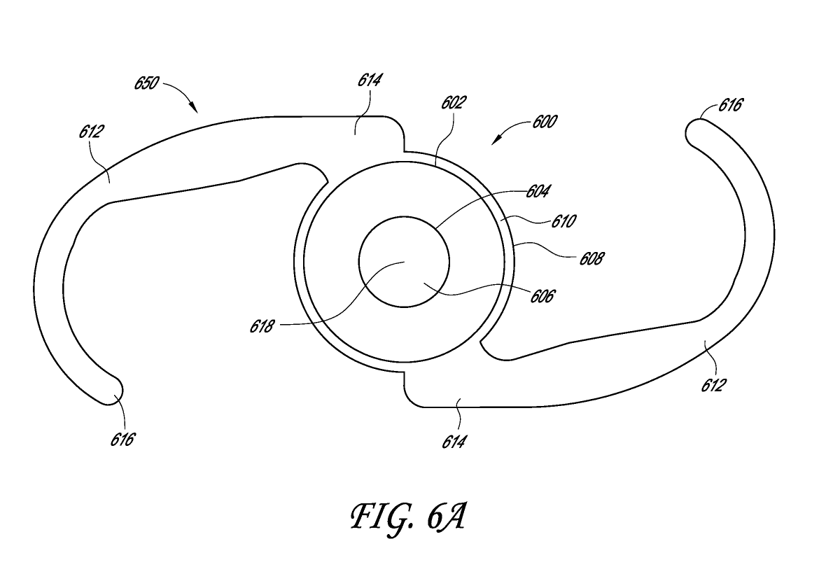

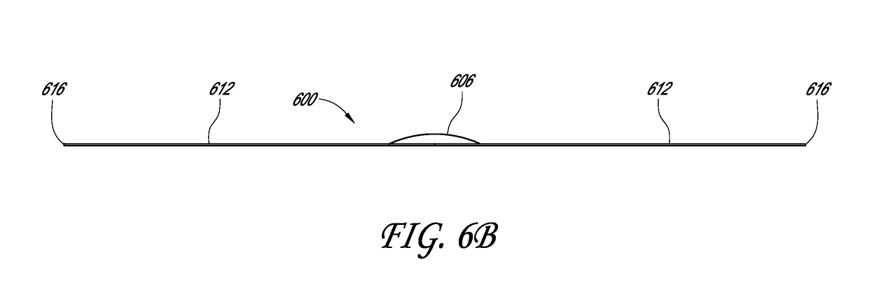

In some embodiments of the ocular device, a mask 500, 600 can be applied to a transparent body 550, 650 to form a non-transmissive region with a small central transmissive un-masked optical aperture, as best illustrated in FIGS. 5-6B. The transparent body 550, 650 can comprise integral haptics 512, 612, and can further be made using any suitable materials, including the materials referred to in this application. Various methods can be used to apply the mask 500, 600 to the transparent body, such as printing, bonding, adhering, etching, or mechanically attaching. For example, the mask 500, 600 can be applied to the body 550, 650 by printing dyes and pigments onto the body's 550, 650 surface, or through using other materials that can be used to create non-transmissive regions as discussed further below. The mask 500, 600 can also be embedded with the transparent body 550, 650. In certain embodiments, the outer periphery 502, 602 of the mask 500, 600 coincides with the outer periphery 508, 608 of the transparent body 550, 650. In the illustrated embodiments, the outer periphery 502, 602 of the mask 500, 600 does not extend all the way out to the outer periphery 508, 608 of the transparent body 550, 650, creating a transparent ring 510, 610. In some embodiments, the transparent body 550, 650 onto which the mask 500, 600 is applied is all or nearly all transparent and has no optical power. In some embodiments, such as in FIG. 6, the transparent body 650 comprises a center portion 606 with optical power.

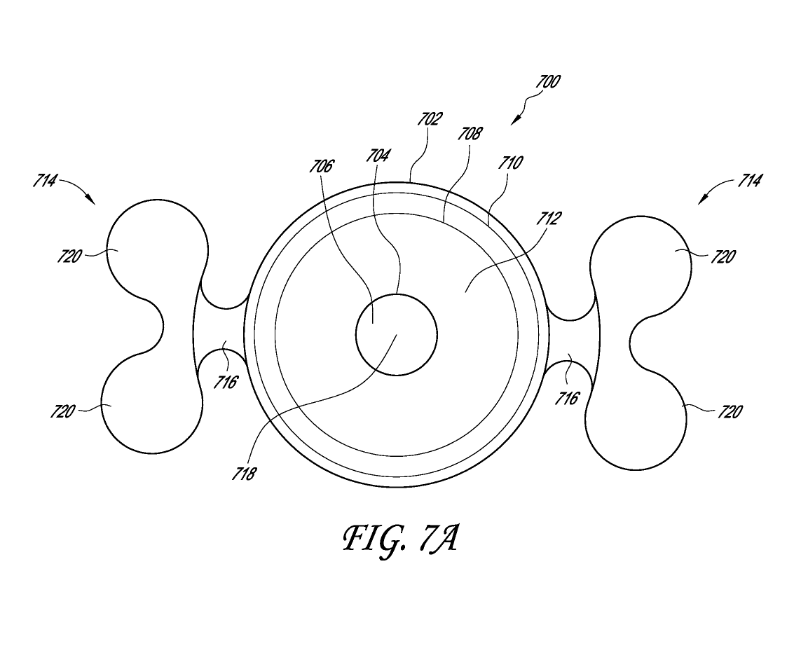

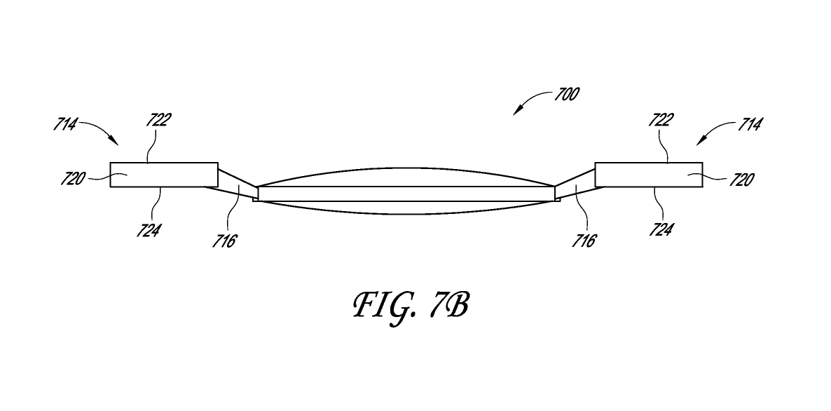

The ocular device can also be implanted in the anterior chamber of a patient's eye, and various retention members can be used in this location, as described in U.S. Patent Publication No. 2011/0040376 (corresponding to U.S. patent application Ser. No. 12/856,492, filed Aug. 13, 2010), which is incorporated by reference herein in its entirety. FIGS. 7A and 7B illustrate one embodiment of an ocular device with haptics 714 for securing the device in the anterior chamber of an eye. In some embodiments, the haptics 714 can be fixed to and can extend generally outwardly from an outer periphery 702 of the mask 700. In certain embodiments, the haptics 714 are positioned on opposing sides of the mask 700. In some embodiments, the haptics 714 can comprise a bridge 716 with at least one footplate 720. The bridge 716 and the footplate 720 can be made using any suitable materials, including the materials referred to in this application. In certain embodiments, the bridge 716 can be made from a flexible material to allow the haptics 714 to be flexed or moved relative to the mask 700. The footplate 720 can be made in a wide variety of shapes and configurations to support the mask 700 within the eye. In some embodiments, like that shown in FIG. 7, the footplate can comprise two or more lobes wherein each lobe has a generally radially outwardly facing concavity. In some embodiments, the footplate 720 can comprise two or more lobes extending generally outwardly from the bridge 716. In some embodiments comprising a footplate 720, the footplate 720 can have a thickness, the thickness being measured from a first surface 722 to a second surface 724 of the footplate 720. The thickness can be measured along an axis generally transverse to a longitudinal axis of the bridge 716. The thickness can be greater than or equal to a diameter of a transmissive region 704 and/or less than or equal to a diameter of the mask 700. In certain aspects, the thickness can be greater than at least half the diameter of the mask, greater than at least three-fourths the diameter of the mask, and/or substantially the same as the diameter of the mask. The thickness of the footplate can be at least about 0.20 mm and/or less than or equal to about 0.30 mm. In some embodiments, the thickness of the footplate 720 can be between about 0.20 mm and about 0.25 mm. In some embodiments with haptics comprising a footplate comprised for attachment in the anterior chamber of the eye, the total diameter of the device including the haptics can be in the range of at least about 10 mm and/or less than or equal to about 14 mm. In some embodiments configured for implantation in the anterior chamber of the eye, the total diameter including the haptics can be at least 11 mm.

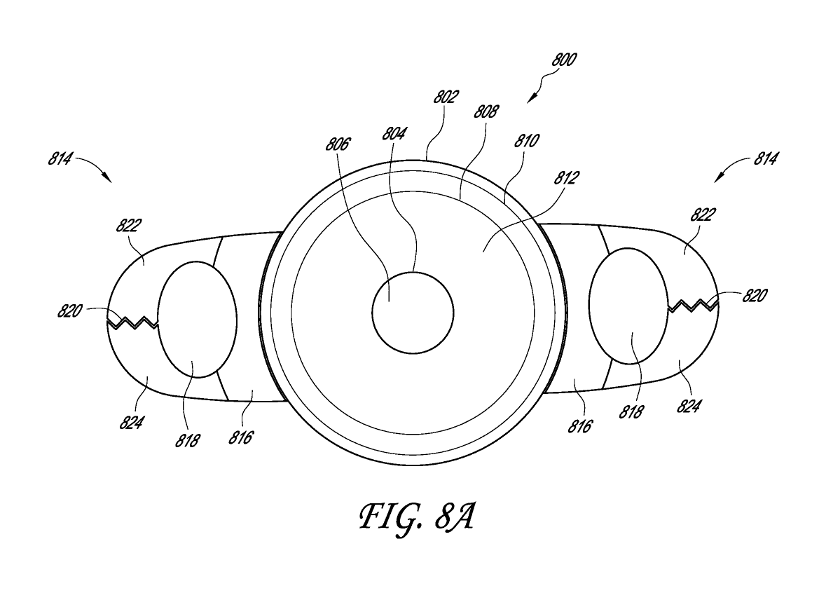

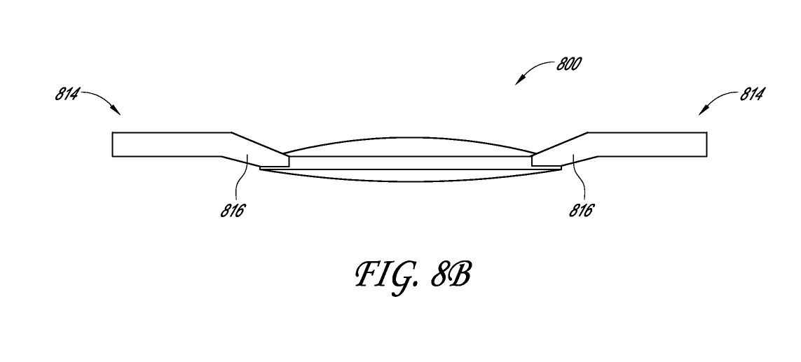

The ocular device of the present application can also be implanted in a position between the IOL and an iris or between the iris and a cornea. In some embodiments, the ocular device can be secured to the iris, and various retention members can be used to secure the device to the iris. FIGS. 8A and 8B illustrate one embodiment of an ocular device with a mask 800 with haptics 814 for securing the device to the iris. In one embodiment, the haptics 814 can extend generally outwardly from an outer periphery 802 of the mask 800 and can be positioned on opposing sides of the mask 800. In some embodiments, each haptic 814 can comprise a proximal portion 816, as well as an upper distal portion 822 and a lower distal portion 824 separated by a part line 820. In some embodiments, the upper and lower distal portions are elastically deformable or resiliently move apart to create a claw for grasping a portion of the iris tissue. The proximal portion 816 of the haptics 814 can be flexible, permitting the haptics 814 to be moved or flexed relative to the mask 800. The claws can be attached to an anterior surface of the iris or a posterior surface of the iris. In some embodiments, the claws are detachably attached to the iris so that the ocular device can be removed or repositioned, depending on the clinical need of a patient. In some embodiments, the claws attach the ocular device to the iris so that the mask 800 is centered on an optical axis of the eye. In some embodiments, the haptics may comprise an opening 818 which can be advantageous for such reasons as reducing the mass of the haptics 814 and allowing improved manipulation of the device during implantation. In some embodiments with haptics 814 configured for attachment to the iris, the total diameter of the device including the haptics 814 can be in the range from about 10 mm to about 14 mm. In some embodiments, the total diameter including the haptics 814 can be at least 11 mm.

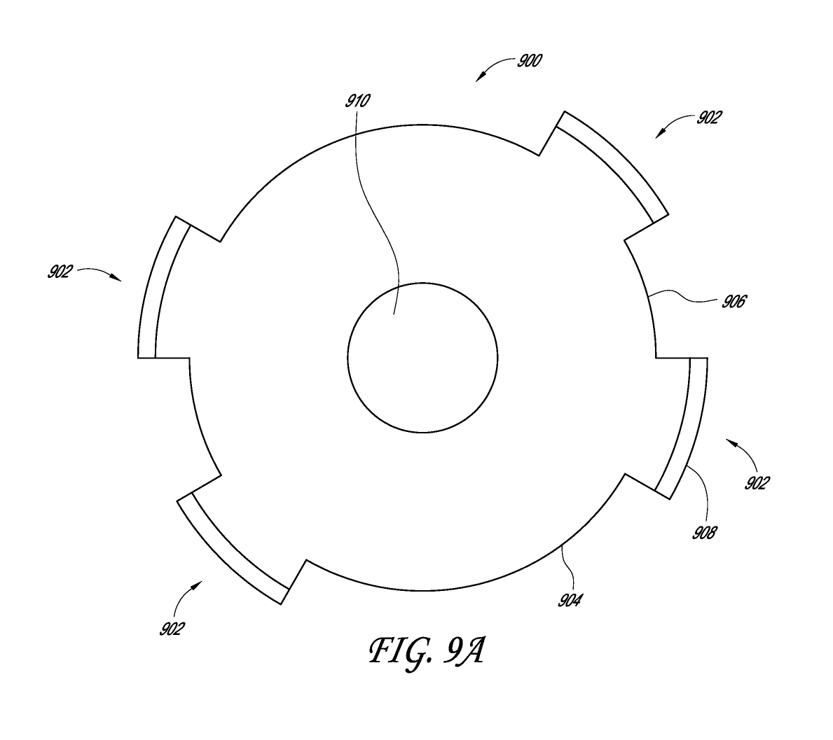

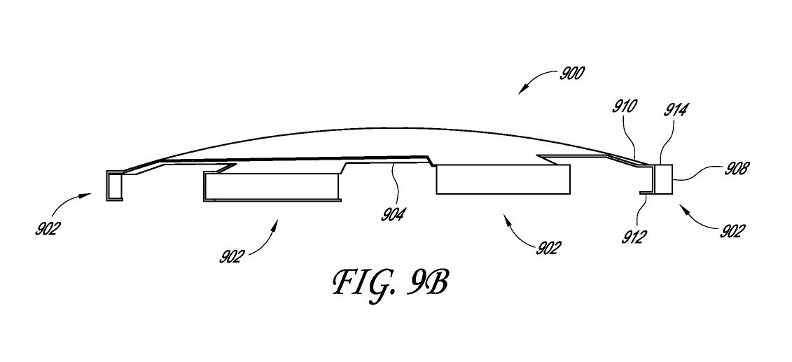

In certain embodiments, the ocular device can be positioned adjacent to an IOL by attaching the device directly to an IOL through one of a variety of methods. Advantageously, the device can be attached to an IOL prior to implantation of the IOL or the device can be attached to an IOL that was previously implanted into the patient's eye. In some embodiments, best illustrated in FIGS. 9A-9B, an ocular device can comprise a mask 900 with one or more clips or hooks 902. In some embodiments, the hooks 902 can be spaced at various positions on or near the outer periphery of the mask 900. In certain embodiments, the hooks 902 are not spaced evenly around the outer periphery of the mask. For example, pairs or groups of hooks 902 can be positioned on generally opposing sides of the mask 902, wherein a relatively small gap 906 can separate the hooks in each pair while a larger gap 904 separates the pairs. Various arrangements of the hooks may be advantageous, depending on the IOL to which the mask 900 will be attached. For example, the larger gap 904 may desirably allow the hooks 902 to be attached to an IOL without interfering with the haptics of the IOL.

Features of some embodiments of the hooks 902 can be best seen in FIG. 9B. The hooks 902 can be made in a variety of shapes and can have a variety of features to provide for attachment of the mask to an IOL. In some embodiments, the hooks 902 can have an outer surface 908 with an outwardly radial convex curvature to correspond to the generally circular periphery of an IOL. In certain embodiments, the hooks 902 can comprise lead-in ramps 910 which can extend generally outwardly from an outer periphery of the mask 900. In some embodiments, the ramps 910 can have a curvature generally consistent with that of the mask 900 and can extend outwardly from the outer periphery of the mask 900. In some embodiments, the ramps 910 can have a curvature extending to a flat portion 914 towards the outer circumference of the hook 902. In some embodiments, the ramps 910 can be planar or flat, such that they are substantially perpendicular to the patient's line of sight. Importantly, the ramps 910 can be made in a variety of sizes and configurations to match the clinical needs of the patient and the configuration of any IOL.

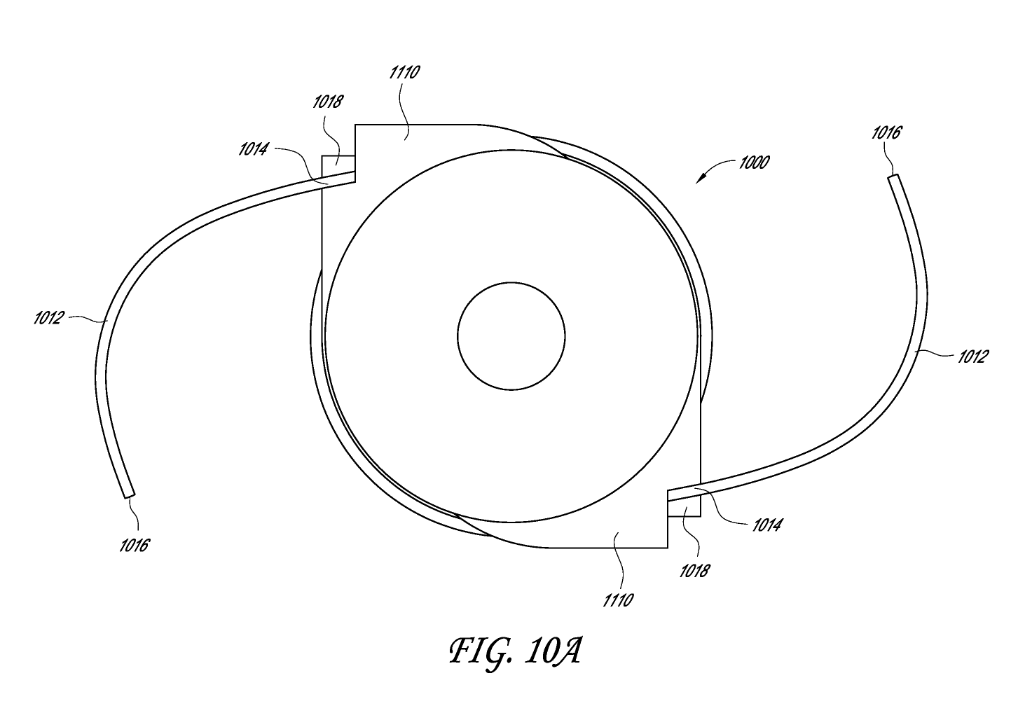

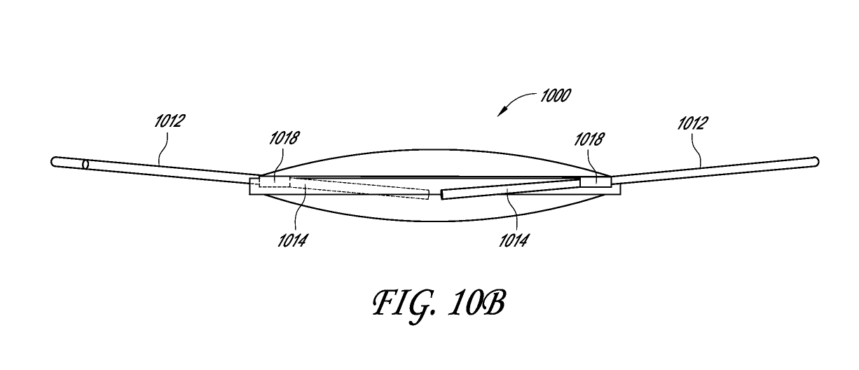

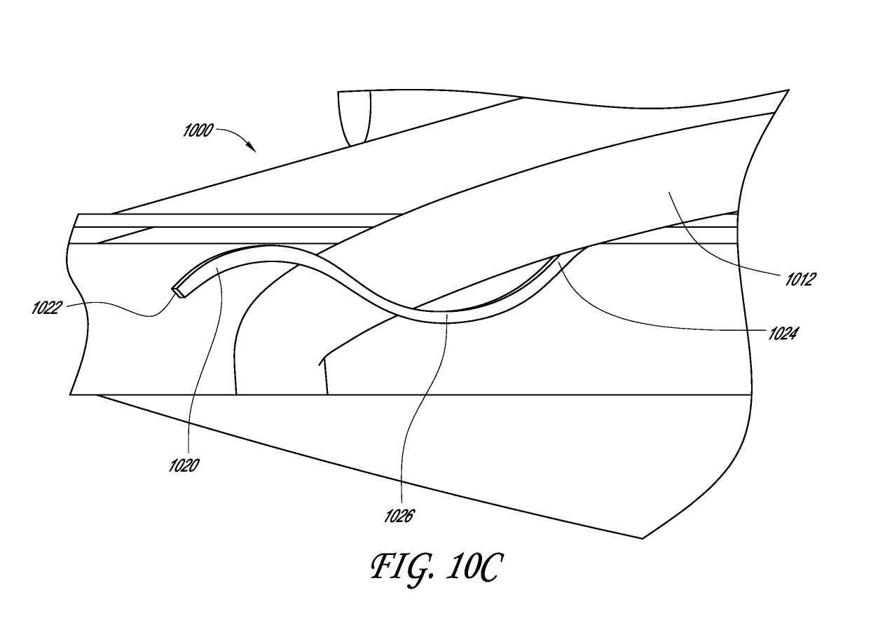

FIGS. 10A-10C illustrate another embodiment of an ocular device with a mask 1000 comprising spring clips 1020. In certain embodiments, spring clips 1020 can attach the mask 1000 to haptics of an IOL. The spring clips 1020 can allow the mask 1000 to be attached to commonly available IOLs, including three-piece IOLs, for example. FIG. 10C best illustrates the details of some embodiments of the spring clips 1020. In the illustrated embodiment, spring clip 1020 has a distal end 1022, a proximal end 1024, and an intermediate section 1026. The proximal end 1024 can attach to a surface or an edge of mask 1000 while the intermediate portion 1026 of the spring clip 1020 can wrap around the haptic 1012 of the IOL, thus attaching the mask 1000 to the IOL.

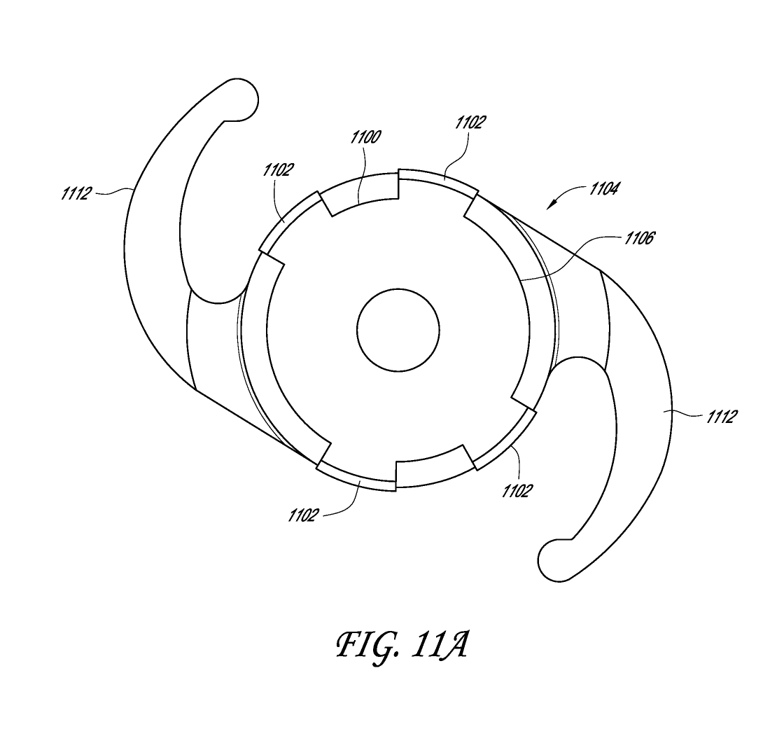

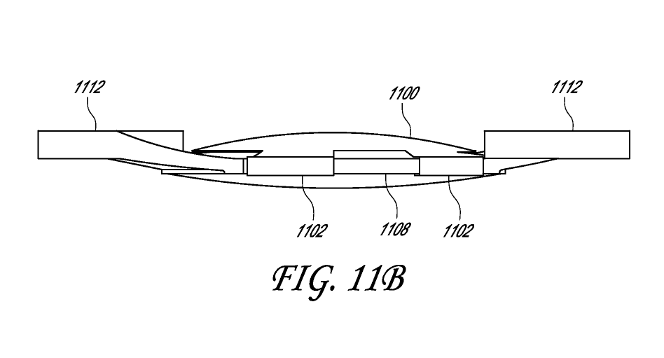

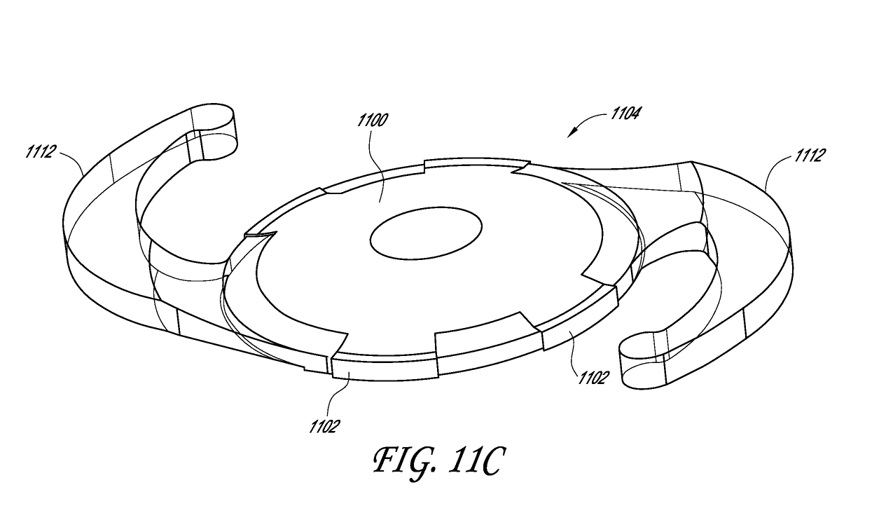

FIGS. 11A-11C illustrate a mask 1100 attached to an IOL 1104. In the embodiment shown, the mask 1100 is attached to a one-piece IOL 1104. As shown, in some embodiments the hooks 1102 can be generally positioned at or near an outer periphery 1106 of the mask 1100. Desirably, the hooks 1102 can be positioned so as not to interfere with the haptics 1112 of the IOL 1104. In some embodiments, the hooks 1102 can wrap around or clip onto the outer edge of 1108 so as to attach the mask 1100 to the IOL 1104.

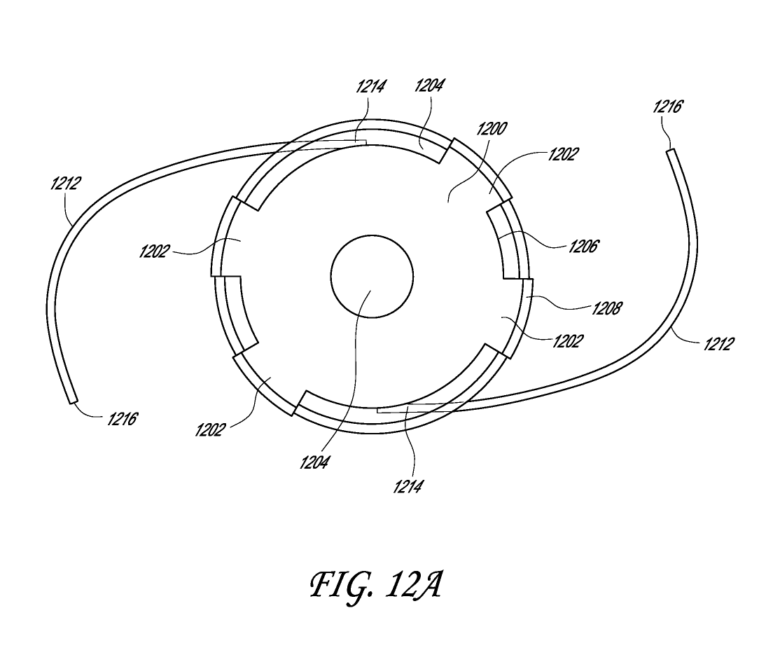

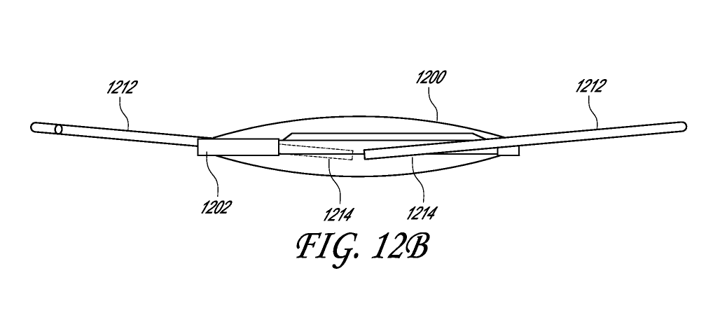

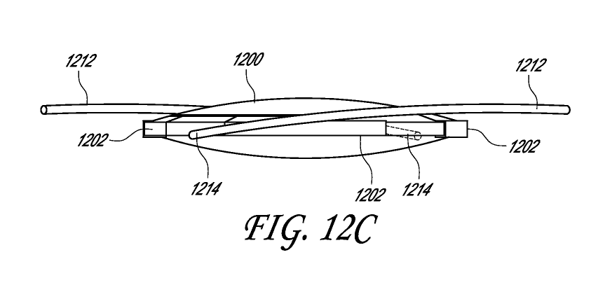

FIGS. 12A-12C illustrate a mask 1200 attached to an IOL 1204. In the embodiment shown, the mask 1200 is attached to a three-piece IOL 1204, although the hooks 1202 of mask 1200 can be configured to attach to any of a variety of IOLs available to patients.

In some embodiments, the ocular device of the present application can move forward and backward along the optical axis inside of an eye. For example, the haptics can support the device within the eye, while also allowing the device to move slightly. Such movement of the device, in some embodiments, may be advantageous, particularly when the device is placed in a position adjacent to an accommodating IOL, which may move forward and backward inside the eye as the patient focuses on certain near or far objects.

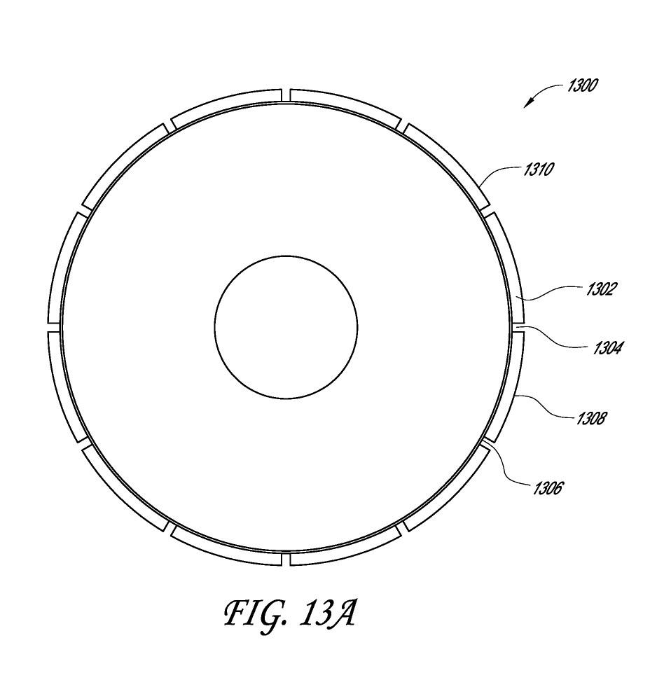

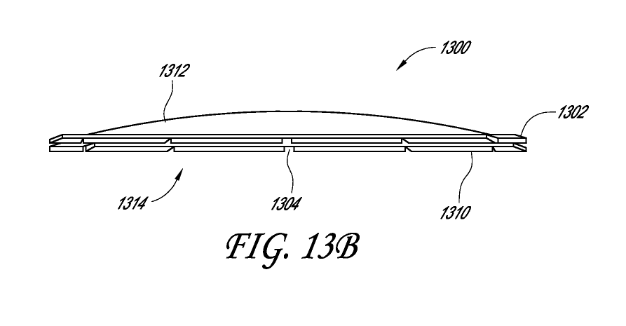

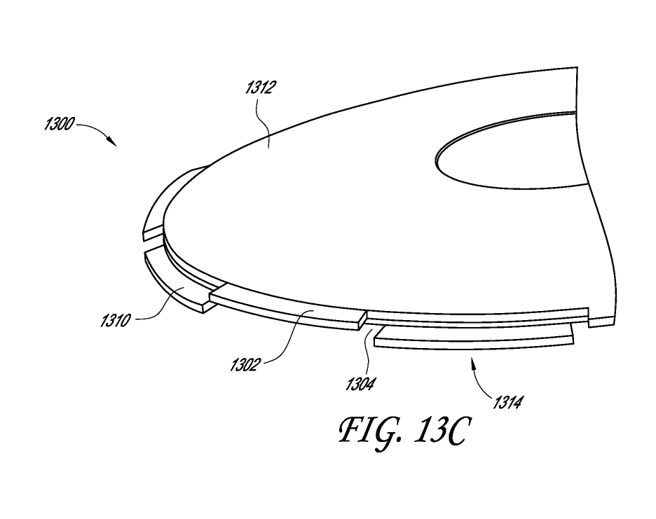

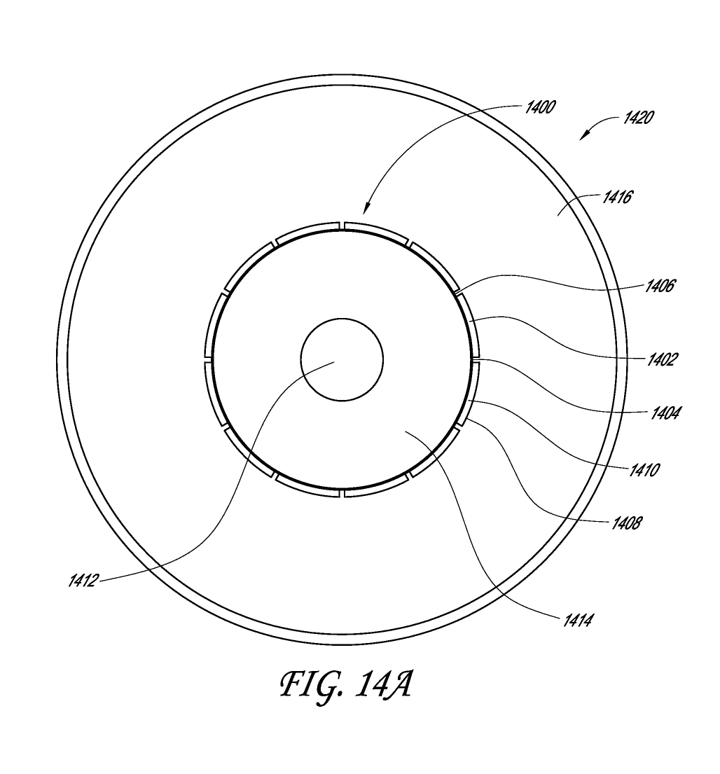

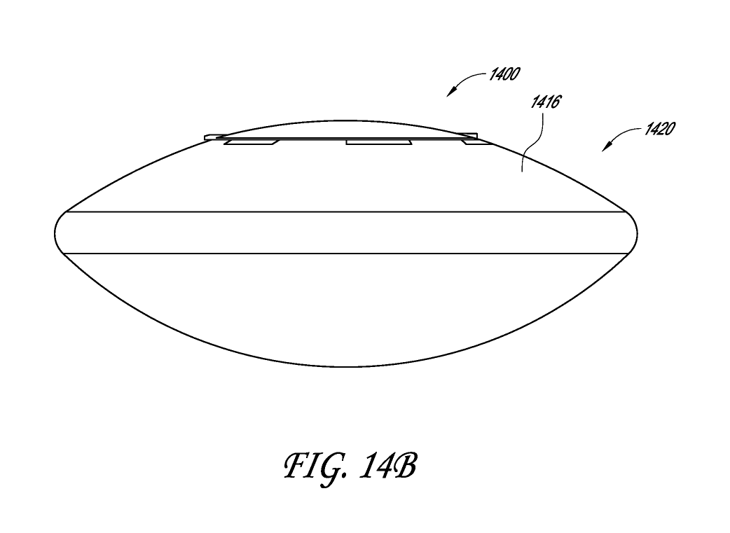

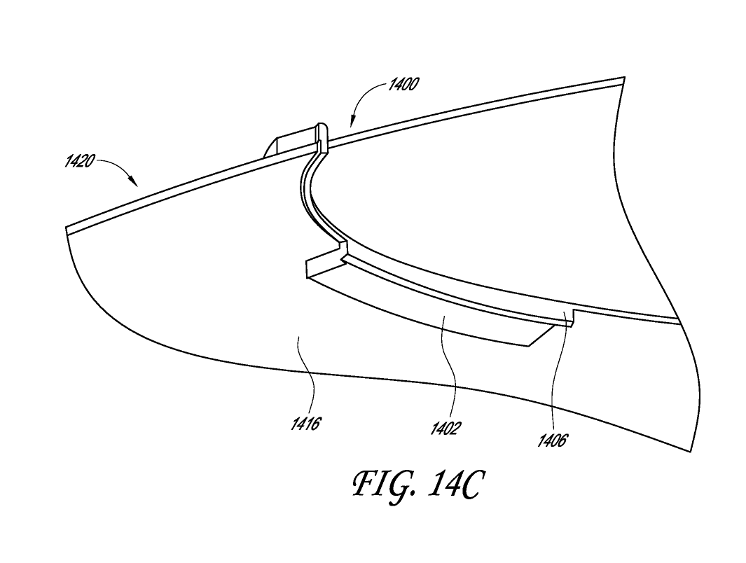

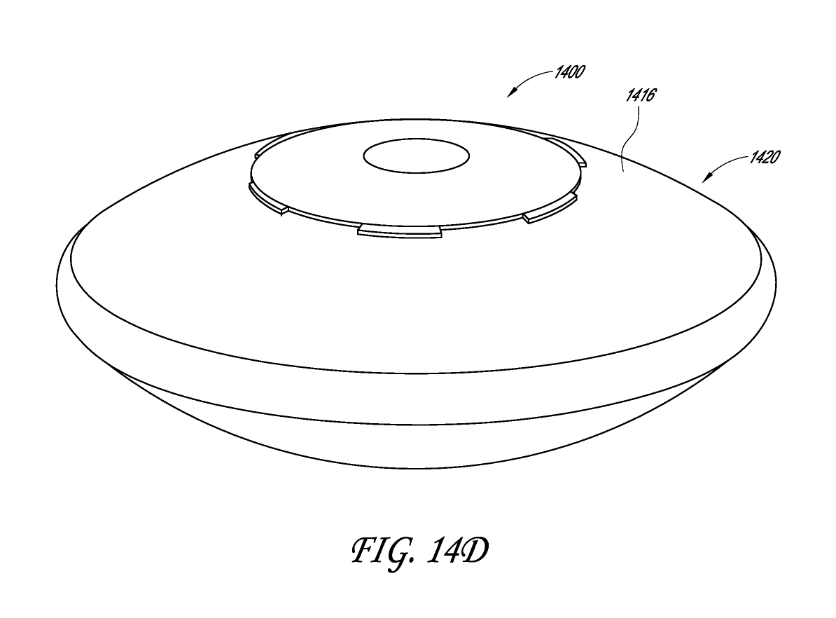

In some embodiments, best illustrated by FIGS. 13A-14D, the device comprises a grooved edge positioned on or near the outer periphery of a mask. The grooved edges can act as retention members configured to support the device within the eye. In some embodiments, the grooved edges of the device are positioned to correspond to the incision made on a lens capsule during a capsulotomy. This embodiment of the device may be particularly desirable when the capsulotomy is performed by an ophthalmic surgical laser, such as a femtosecond laser. In some embodiments, for example, the laser can be programmed to make a substantially circular incision with a particular diameter, and a mask with a corresponding diameter can then be provided. The selected mask can then be inserted within the opening on the lens capsule, and the mask can grip the opening to support the mask within the eye. More particularly, the mask can have an outer periphery with a diameter corresponding to a diameter of a capsulotomy incision, so that the grooved edges near the outer periphery of the mask can be fitted into the capsulotomy incision. Importantly, the mask size and shape can be customized to correspond to a variety of capsulotomy incisions. For example, in some embodiments, the outer periphery of the mask can have a diameter in the range of about 3 mm to about 8 mm as needed to correspond to the capsulotomy incision. In some embodiments, an inner periphery surrounding an aperture of the mask can have a diameter in the range of about 0.5 mm to about 1.8 mm.

The illustrated embodiment of FIGS. 13A-13C comprises a mask with grooved edges, or protrusions positioned at or near the outer periphery 1306 of the mask 1300. In some embodiments, the mask 1300 has anterior protrusions 1302 extending generally outwardly from or near an anterior surface 1312 of the mask 1300, while in some embodiments the mask has posterior protrusions 1310 extending generally outwardly from or near a posterior surface 1314 of the mask 1300. In some embodiments, the mask 1300 comprises both anterior and posterior protrusions 1302, 1310 extending generally outwardly from the mask 1300. The anterior and posterior protrusions 1302, 1310 can be arranged on the mask in any of a variety of configurations to facilitate attachment to a patient's lens capsule. For example, in some embodiments, the anterior and posterior protrusions 1302, 1310 can be arranged in an alternating pattern wherein each anterior protrusion 1302 is separated from a next anterior protrusion 1302 along the outer periphery 1306 of the mask 1300 by at least one posterior protrusion 1310, as best shown in FIG. 13C. In some embodiments, the anterior and posterior protrusions 1302, 1310 extend outwardly from the mask 1300 such that at least one of the protrusions 1302, 1310 is substantially perpendicular to the patient's line of sight. In certain embodiments, at least one of the protrusions 1302, 1310 can be generally planar. In some embodiments, at least one of the protrusions 1302, 1310 can have a curvature to substantially correspond with a curvature of a surface of a patient's lens capsule. In some embodiments, the protrusions 1302, 1310 surround the entire outer periphery of the mask 1306. In some embodiments, at least one protrusion 1302, 1310 is separated from an adjacent protrusion 1302, 1310 by a gap 1304. The gap 1304 can be a variety of sizes in order to allow for attachment of the mask to a patient's lens capsule.

FIGS. 14A-14D illustrate one embodiment of a mask 1400 similar to the mask illustrated in FIG. 13A-C, except that the mask 1400 is attached to a patient's lens capsule 1420. As illustrated, the mask 1400 can be placed adjacent to an IOL by positioning the mask 1400 within the capsulotomy opening on the anterior surface of the lens capsule 1402 by protrusions 1402, 1410.

IV. Ocular Device with a Mask Configured to Reduce Diffraction Patterns

In certain embodiments, the mask includes a transmissive zone or region, and a non-transmissive zone or region. For example, in FIG. 1, the mask 100 comprises a transmissive region 106 and a non-transmissive region 108. In some embodiments, the transmissive region can be located at least partially within an outer region of the mask. In some embodiments, the transmissive region can be completely surrounded by the outer region of the mask. In some embodiments, the transmissive zone is desirably centrally located within the outer region of the mask. In one embodiment, the geometric center of the transmissive region and the geometric center of the mask coincide, e.g., at the central axis of the mask.

In some embodiments, the transmissive region can be implanted at least partially in an optical zone of the eye, such that light entering through the cornea passes through the transmissive region before reaching the retina. In certain embodiments, the transmissive region can be substantially centered on the optical axis of the eye, such as the line of sight and an axis passing through the center of the entrance pupil and the center of the patient's eye. In some embodiments, the transmissive region can transmit a majority of light in the visible range. In one embodiment, the transmissive region transmits all or nearly all of the light in the visible range. In one embodiment, the transmissive region transmits at least about 90% of the light in the visible range. In some embodiments, the transmissive region transmits at least about 80% of the light in the visible range. In some embodiments, the transmissive region can be completely transparent and can transmit all of the light in the visible range.

The transmissive region can be sized to cover a substantial portion of the optical zone of the IOL in one embodiment. For example, the transmissive region can cover more than half of the optical zone when the iris is fully dilated. In some embodiments, the transmissive region can cover substantially the entire optical zone when the iris is fully dilated. In some embodiments, the transmissive region can cover the entire optical zone when the iris is fully dilated. In some embodiments, the transmissive region can cover more than half of the optical zone when the iris is fully constricted. In some embodiments, the transmissive region can cover substantially the entire optical zone when the iris is fully constricted. In another embodiment, the transmissive region can cover the entire optical zone when the iris is fully constricted.

A transmissive region can be formed with any suitable transverse dimension, e.g. a diameter in the range of about 0.5 mm to about 1.8 mm. In one embodiment, the transmissive zone can have a transverse dimension of at least about 0.7 mm. Some embodiments can include a smaller transmissive region, e.g., a diameter less than 1.3 mm. In one embodiment, the mask can have a transmissive region with a transverse dimension greater than that which would produce a pinhole effect. Such an arrangement allows more light to reach the retina which may be advantageous, particularly in dark conditions or while driving at night. Such an arrangement may also be particularly advantageous for patients who do not have difficulty with accommodation.

In addition to a transmissive region, some embodiments also have a non-transmissive region as mentioned above, and as further discussed below. In some embodiments, a relatively sharp boundary or demarcation can be provided between an outer periphery of the transmissive region and the inner periphery of the non-transmissive region. For example, in some embodiments, the outer periphery of the transmissive region and the inner periphery of the non-transmissive region coincide, and create a sharp boundary between the two regions. In some embodiments, the mask can display a more gradual change in opacity from the transmissive region to the non-transmissive region. For example, various apodization techniques can be applied to portions of the mask, such that there is a gradual increase in opacity between the transmissive region and the non-transmissive region, examples of which are described in U.S. Pat. No. 7,628,810, issued Dec. 8, 2009, and U.S. Patent Publication 2012/0143325 (corresponding to U.S. patent application Ser. No. 13/390,080, filed Feb. 10, 2012), both of which are incorporated by reference herein in their entireties. In one embodiment, an apodization technique can also be used to create a sharp boundary between a transmissive region and a non-transmissive region where the sharp boundary between the regions varies in distance from the central axis of the mask, e.g., the boundary is undulating or wavy. A variety of other apodization techniques are set forth in U.S. Pat. Nos. 5,662,706; 5,905,561; and 5,965,330, which are all incorporated by reference herein in their entireties.

In some embodiments, the non-transmissive region can be defined by an outer periphery and an inner periphery. As illustrated in FIG. 1, in some embodiments, the outer periphery of the non-transmissive region 108 can coincide with the outer periphery 102 of the mask 100. In some embodiments, the inner periphery of the non-transmissive region can coincide with the outer periphery of the transmissive region.

The non-transmissive region can have different degrees of opacity. In some embodiments, the non-transmissive region can block all of visible light or substantially all of visible light incident on the anterior surface of the non-transmissive region. In one embodiment, the non-transmissive region blocks more than 50% of the visible light incident on the anterior surface of the non-transmissive region. In some embodiments, the non-transmissive region blocks at least about 60% of the visible light incident on the anterior surface of the non-transmissive region. In some embodiments, the non-transmissive region blocks at least about 70% of the visible light incident on the anterior surface of the non-transmissive region. In some embodiments, the non-transmissive region blocks at least about 80% of the visible light incident on the anterior surface of the non-transmissive region. In some embodiments, the non-transmissive region blocks about 90% or 95% or more of the visible light incident on the anterior surface of the non-transmissive region. In an alternate embodiment, the non-transmissive region is an opaque region that transmits no more than 20% of the visible light incident on the anterior surface of the non-transmissive region. In some embodiments, the non-transmissive region may be completely opaque.

The opacity of the non-transmissive region may also vary in different parts of the mask. For example, in certain embodiments, the opacity near the outer periphery or inner periphery of the mask can be less than a central part of the non-transmissive region as described in U.S. Pat. No. 7,628,810, issued Dec. 8, 2009, and U.S. application Ser. No. 13/390,080, filed Feb. 10, 2012, both of which are incorporated by reference herein in their entireties. The opacity in different parts of the non-transmissive region may transition abruptly or have a gradient transition. Additional examples of opacity transitions can be found in U.S. Pat. Nos. 5,662,706, 5,905,561 and 5,965,330, which are incorporated in their entirety herein by reference.

Opacity of the non-transmissive region can be achieved in any of several different ways. For example, in some embodiments, the material used to make a mask may be naturally opaque. In some embodiments, the material used to make the mask 100 may be substantially clear, but treated with a dye or other pigmentation agent to render the non-transmissive region substantially opaque. In some embodiments, the dye is selected from those providing low transmission in some wavelengths, and greater transmission in other wavelengths. Such a dye may be advantageous for use with various diagnostic technique and instruments. For example, common contemporary diagnostic techniques can utilize Scanning Laser Ophthalmoscopy/Optical Coherence Tomography (SLO/OCT), which typically have an illumination source in the near-infrared (NIR) range e.g. 850 nm. Therefore, it may be desirable for the mask to block visible light (e.g. wavelengths between about 400 nm and about 700 nm) while retaining a high transmission at a diagnostic instrument's operational wavelengths of NIR light. In one embodiment, the non-transmissive region can be configured of a material absorbent in an appropriate range of wavelengths to provide opacity in visible light, but also transparent to NIR light, as disclosed in U.S. application Ser. No. 13/691,625, filed Nov. 30, 2012, and titled “Ocular Mask Having Selective Spectral Transmission,” which is incorporated by reference herein in its entirety. Such a material composition may desirably minimize the visibility of the mask during an examination using diagnostic instruments with infrared light sources.

In some embodiments, the non-transmissive region can comprise a light absorbing material embedded within or combined with another material. For example, the non-transmissive region can be formed by mixing together a suitable polymer material and sufficient quantity of an opacification agent. Such a mixture may provide adequate absorption of light and prevent noticeable refractive difference across the transition from the transmissive region to the non-transmissive region. As discussed above, carbon is one example of a suitable opacification agent, although others can be used. In one embodiment, carbon can include carbon black and/or small, e.g., submicron, powdered carbon particles.

In some embodiments, the surface of the mask may be treated with a particulate deposited thereon. For example, a particulate of titanium, gold or carbon may be deposited on the surface of the mask provide opacity. In some embodiments, the particulate may be encapsulated within the interior of the mask. Some embodiments employ different ways of controlling light transmission through the mask. In one embodiment, the mask may comprise a gel, such as hydrogel or collagen, or other suitable material. The gel within the mask can further include a particulate suspended within the gel. Examples of suitable particulate are gold, titanium, and carbon particulate.

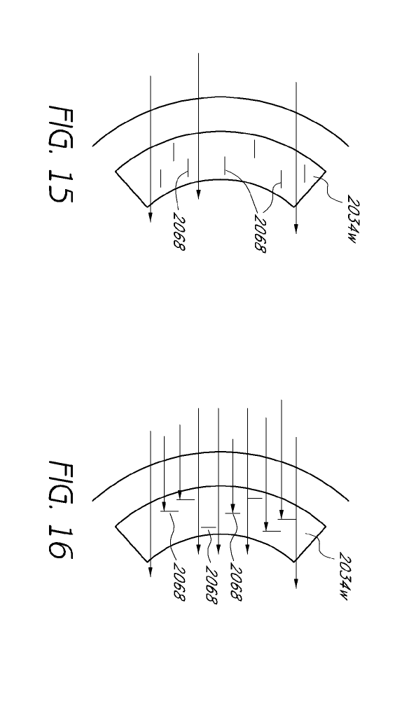

FIGS. 15 and 16 illustrate one embodiment where a mask 2034w comprises a plurality of nanites 2068. “Nanites” are small particulate structures that have been adapted to selectively transmit or block light entering the eye of the patient. The particles may be of a very small size typical of the particles used in nanotechnology applications. The nanites 2068 are suspended in the gel or otherwise inserted into the interior of the mask 2034w, as generally shown in FIGS. 15 and 16. The nanites 2068 can be preprogrammed to respond to different light environments.

Thus, as shown in FIG. 15, in a high light environment, the nanites 2068 turn and position themselves to substantially and selectively block some of the light from entering the eye. However, in a low light environment where it is desirable for more light to enter the eye, nanites may respond by turning or be otherwise positioned to allow more light to enter the eye, as shown in FIG. 16.

Nano-devices or nanites are crystalline structures grown in laboratories. The nanites may be treated such that they are receptive to different stimuli such as light. In accordance with one aspect of certain embodiments, the nanites can be imparted with energy where, in response to a low light and high light environments, they rotate in the manner described above and generally shown in FIG. 16.

Nanoscale devices and systems and their fabrication are described in Smith et al., “Nanofabrication,” Physics Today, February 1990, pp. 24-30 and in Craighead, “Nanoelectromechanical Systems,” Science, Nov. 24, 2000, Vol. 290, pp. 1502-1505, both of which are incorporated by reference herein in their entirety. Tailoring the properties of small-sized particles for optical applications is disclosed in Chen et al. “Diffractive Phase Elements Based on Two-Dimensional Artificial Dielectrics,” Optics Letters, Jan. 15, 1995, Vol. 20, No. 2, pp. 121-123, also incorporated by reference herein in its entirety.

In some embodiments, the surface of the mask can be treated physically or chemically (such as by etching) to alter the refractive and transmissive properties of the mask and to alter the transmission of light.

Although in certain embodiments the non-transmissive region is a peripheral region and is described as an “opaque” region, any construction that substantially prevents light from passing through the region could provide at least some of the advantages described herein, such as reducing glare or other distracting visual effect caused by the ocular device. Other optical phenomena that can be used to prevent transmission of light at the non-transmissive region are described in U.S. Pat. No. 6,554,424, which is incorporated by reference herein in its entirety. Such phenomena can include one or more of reflection of light in the non-transmissive region, diffraction of light in the non-transmissive region, and scattering of light in the non-transmissive region, alone or in combination with light absorption to provide at least one of the advantages described herein.

The non-transmissive region can provide an advantage of preventing distracting visual effects from being visible to the patient, in some embodiments. For example, the non-transmissive region can block enough light to eliminate distracting visual effects at a periphery of the ocular device. Some configurations of the non-transmissive region can also reduce glare and other distracting visual effects at a boundary between the ocular device and the IOL, particularly the IOL portion that resides adjacent to the outer periphery of the mask. Glare can occur due to the difference in refraction of the light that passes through the ocular device and the light that passes through the adjacent IOL and not through the ocular device. Such refractive difference can be significant enough to be noticed by a patient, and thus can be distracting. In some embodiments, glare can be reduced by making the width of the non-transmissive region large enough to provide sufficient distance between the light passing through the transmissive region and the light passing through the lens outside of the ocular device.

As mentioned above, the non-transmissive region can be configured to reduce a noticeable difference in refraction of light that passes through the transmissive region of the device and a central optical zone of the IOL and light that passes through the optical zone of the IOL but around the device, e.g., outside the outer periphery of the device. In one embodiment, the non-transmissive region can be annular and can surround the transmissive region. In one embodiment, the non-transmissive region comprises an annular shape in which at least one periphery thereof is substantially circular. In some embodiments, the non-transmissive region has an inner periphery and an outer periphery, at least one of which is circular. In some embodiments, an annular non-transmissive region can also have an irregular or wavy inner or outer periphery that varies in distance from the central optical axis of the device. In some embodiments, the inner periphery of the non-transmissive region may coincide with an outer periphery of the transmissive region.

In some embodiments, the non-transmissive region is generally annular. The non-transmissive region may have a transverse dimension that can include the width of the annulus. In certain embodiments, the non-transmissive region can have a transverse dimension that is approximately two times the width of the transmissive region. In certain embodiments, the inner periphery of the non-transmissive region can have a diameter between about 0.5 mm and about 1.8 mm. In one embodiment, the inner periphery of the non-transmissive region can have a diameter of at least about 1.0 mm. In one embodiment, the combined width of the two portions of the non-transmissive region on opposite sides of the transmissive region is about 1.5 mm or more. In some embodiments, the combined width of the two portions of the non-transmissive region on opposite sides of the transmissive region is about 1.3 mm or more. In some embodiments, the combined width of the two portions of the non-transmissive region on opposite sides of the transmissive region is at least about 0.8 mm or more. In some embodiments, a transverse dimension of the transmissive region is greater than a transverse dimension of the non-transmissive region. In some embodiments, a transverse dimension of the transmissive region is less than a transverse dimension of the non-transmissive region. In one embodiment, the mask can be configured such that an inner periphery of the non-transmissive region has a transverse dimension that is greater than that which would produce a pinhole effect. Such an arrangement allows more light to enter the eye and may be advantageous, particularly in dark conditions. Such an arrangement may also be particularly advantageous for patients that do not have difficulty with accommodation.

In some embodiments, the non-transmissive region may have a transverse dimension sufficient to extend to a projection of a pupil of the eye. For example, the width of the non-transmissive region extending across the transmissive region can be about 8 mm or more. Here, the non-transmissive region can substantially reduce glare by preventing light from being transmitted through adjacent corneal tissue.

### V. Methods of Implanting an Ocular Device

The ocular device of the present invention can be surgically implanted through a wide variety of methods. The device can be applied to the eye in any manner. For example, the ocular device can be joined to an IOL prior to surgical implantation, so that the device and the IOL can be implanted simultaneously during a single procedure. The device and IOL can also be implanted sequentially, in any order, during a single procedure. In some embodiments, the device can be implanted adjacent to a patient's previously implanted IOL. Advantageously, a surgical incision of no more than about 4.5 mm, no more than about 4.0 mm, and in some implementations, no more than about 3.55 mm is required to insert the device in any of the few sample procedures described in more detail below. In some embodiments, a central axis of the device is positioned within the eye to be in line with the eye's optical axis.

The ocular device can also be implanted in any location within the eye, including the anterior chamber or the posterior chamber. For example, the device can be implanted in the posterior chamber by attaching the device to the ciliary sulcus. As a further example, the device can be implanted within an eye's lens capsule or mounted on an outer surface of the lens capsule. The device can be positioned adjacent to an IOL. The ocular device can be implanted adjacent to any type of IOL, including monofocal IOLs, multifocal IOLs, and accommodating IOLs.

### A) Ocular Device and IOL Inserted Simultaneously During Single Procedure

In one method of implantation, an ocular device and an IOL can be inserted simultaneously into an eye through one incision during a single procedure. To use this implantation method, the device and the IOL can be joined together at some time prior to the procedure to form an IOL construct. For example, the device and the IOL can be joined during the manufacturing process. In another example, the device and the IOL can be joined by a surgeon or other appropriate individual at the clinical site prior to the surgery. Further, the device and IOL can be joined by use of a machine during the manufacturing process, or the device and IOL can also be joined by any individual using a tool specially adapted for joining the device and IOL. The device and the IOL can be permanently joined. In some embodiments, the device and the IOL can be only temporarily joined, so that at some time after insertion the device and IOL are no longer joined. Where the device and IOL are temporarily joined, the device and IOL can be separately removable if the patient needs one of the implants removed.

The device and IOL can be joined to each other through various methods. In one embodiment, the device has its own haptics and the IOL has its own haptics. In one embodiment, some or all of the haptics of the device can be formed into one or two or more hooks or clips and can be used to attach the device to the IOL. In some embodiments, some or all of the haptics of the lens can be used to join the lens to the device. As described above, the shape and curvature of the device and IOL may correspond, further improving attachment and positioning of the device adjacent to the IOL. In some embodiments, an adhesive can be used to join the device to the IOL.

After the device and IOL are joined to form an implantable body, the implantable body can then be folded, rolled, or otherwise deformed for insertion through a small incision in an eye. For example, the body may be implanted by rolling up the body and inserting the rolled-up body into a tube. The tube is then inserted into an incision in the eye, and the body is ejected out of the tube deployed within the eye. Using this method, the body can be implanted within the lens capsule after removal of the natural lens. Using this method, the body can also be implanted in the anterior chamber, posterior chamber, and can also be coupled with or attached to the ciliary sulcus (sometimes referred to as “sulcus-fixated”).

Once inserted into the eye, the shape memory material or other properties of the IOL component of the body will cause the body to unroll and expand. The body can be positioned and fixed in place using any surgical technique used to position and fix IOLs. One or more haptics of the IOL can be used to secure the body to the eye. In some embodiments, one or more haptics of the ocular device can be used to secure the body to the eye. In some embodiments, haptics from both the IOL component and the device component can be used to secure the body to the eye. Surgeons and patients may find this procedure advantageous, as it requires only one incision and one implantation procedure to position both an IOL and the ocular device of the present application.

When joining the device and the IOL together, the retention members of the device can be rotationally offset from the retention members of the IOL. For example, any connectors or haptics extending from the periphery of the device may have a greater thickness than the device itself. For example, in the embodiment of FIG. 1, the mask 100 can have a thickness of greater than zero to about 0.5 mm as discussed above. A connector 120 can join the mask 100 to the haptic 112, and the connector 120 in some embodiments can have a thickness of about 0.1 mm to about 0.35 mm. Thus, in some embodiments of the device, the connectors or haptics extending from the mask may have a greater thickness than the mask portion. In such cases, rotationally offsetting the connectors or haptics of the device from those of the IOL may be advantageous.

### B) Ocular Device and IOL Inserted Sequentially During Single Procedure

In one method of implantation, an ocular device and an IOL can be inserted sequentially into an eye through one incision during a single procedure. It is envisioned that the mask and the IOL can be inserted in any order, depending on the surgeon's preference and the clinical characteristics of the patient. For example, the surgeon can insert the IOL, position and secure the IOL, and then insert the device, and position and secure the device. In some embodiments, the surgeon can insert the device, and position and secure the device, and then insert the IOL, and position and secure the IOL. The surgeon can also insert both the IOL and the device, in any order, prior to positioning and securing the IOL and the device, in any order.

The IOL can be implanted through any suitable procedure. The device can also be implanted through any suitable method. For example, the surgeon can slide the device through the incision and into place, without rolling or otherwise deforming the device. If desired, the device can be folded, rolled, or otherwise deformed for insertion through a small incision in an eye. The device may then be inserted into a restraint, such as a tube. The tube is then inserted into an incision in the eye, and the device is ejected out of the tube and deployed within the eye. Using this method, the device can be implanted within the lens capsule adjacent to an IOL. Using this method, the device can also be implanted in the anterior chamber, posterior chamber, and can also be coupled with or attached to the ciliary sulcus (sometimes referred to as “sulcus-fixated”).

In one embodiment, both the device and IOL can have retention members, such as haptics or hooks or clips or protrusions, for example. Thus, the device and the IOL can be secured and positioned in the eye in a variety of ways. For example, the IOL can be secured to a portion of the eye and the device can be secured to the IOL. In certain aspects, the IOL can be secured to a portion of the eye and the device can be separately secured to any portion or structure of the eye. In certain aspects, the device can be secured to a portion of the eye and the IOL can be secured to the device. When positioning the device and the IOL within the eye, the retention members of the device can be rotationally offset from those of the IOL. Advantageously, the device can be removed from the eye if the patient needs to have the device removed.

### C) Ocular Device Inserted Adacent to Previously Implanted IOL

In one method of implantation, an ocular device can be inserted into an eye in a position adjacent to a previously implanted IOL. This method is advantageous in that it permits a patient who already has an IOL to receive the device during a separate, later procedure. Implantation of the device can be accomplished at least one day, at least one month, or at least one year or more following implantation of the IOL.

The device can be implanted through any suitable method. For example, the surgeon can slide the device through the incision and into place, without rolling or otherwise deforming the device. If desired, the device can be folded, rolled, or otherwise deformed for insertion through a small incision in an eye. The device may then be inserted into a restraint such as a tube. The tube is then inserted into an incision in the eye, and the device is ejected out of the tube and deployed within the eye. Using this method, the device can be implanted within the lens capsule adjacent to an IOL. Using this method, the device can also be implanted in the anterior chamber, posterior chamber, and can also be coupled with or attached to the ciliary sulcus (sometimes referred to as “sulcus-fixated”).

The surgeon can use the retention members, such as haptics or hooks or clips, of the device to secure the device in place. The device can be secured to any appropriate structure in the eye. When positioning the device in the eye, each retention member of the device can be rotationally offset from those of the IOL. Advantageously, the device can be removed from the eye if the patient needs to have the device removed.

Although the ophthalmic devices disclosed herein have been disclosed in the context of certain embodiments and examples, it will be understood by those skilled in the art that the ophthalmic devices extend beyond the specifically disclosed embodiments to other alternative embodiments and/or uses of the embodiments and certain modifications and equivalents thereof. It should be understood that various features and aspects of the disclosed embodiments can be combined with or substituted for one another in order to form varying ophthalmic devices. Accordingly, it is intended that the scope of the soap dispenser herein-disclosed should not be limited by the particular disclosed embodiments described above, but should be determined only by a fair reading of the claims that follow.

1. An intraocular device comprising:
a mask configured to increase the depth of focus of a patient, the mask comprising:
an aperture configured to transmit substantially all visible light;
a non-transmissive region surrounding the aperture, the non-transmissive region configured to be substantially opaque to visible light; and
one or more connectors configured to attach the mask to an intraocular lens.

2. The intraocular device of claim 1, wherein the one or more connectors comprise one or more spring clips.

3. The intraocular device of claim 1, wherein the one or more connectors comprise one or more hooks.

4. The intraocular device of claim 3, wherein at least a portion of each hook includes a curvature generally consistent with a curvature of the mask.

5. The intraocular device of claim 1, wherein the one or more connectors are configured to attach to one or more haptics of the intraocular lens.

6. The intraocular device of claim 1, wherein the one or more connectors are configured to engage an outer periphery of the intraocular lens.

7. The intraocular device of claim 1, wherein the one or more connectors are positioned at an outer periphery of the mask.

8. The intraocular device of claim 1, wherein the one or more connectors include at least two connectors spaced around an outer periphery of the mask.

9. The intraocular device of claim 1, wherein the mask further comprises a plurality of holes disposed in the non-transmissive region, the plurality of holes positioned at irregular locations to reduce visible diffraction patterns due to the transmission of visible light through the holes.

10. The intraocular device of claim 1, wherein at least a portion of the non-transmissive region includes a texturized surface.

11. The intraocular device of claim 10, wherein the portion of the non-transmissive region is at least about 50% of the non-transmissive region.

12. The intraocular device of claim 10, wherein the texturized surface includes a surface roughness of less than about 125 microinches.

13. The intraocular device of claim 1, wherein the mask further comprises a substantially transparent outer region surrounding at least a portion of the non-transmissive region.

14. The intraocular device of claim 1, wherein the mask further comprises nanites configured to selectively transmit light.

15. The intraocular device of claim 1, wherein the one or more connectors are configured to removably attach the mask to the intraocular lens.

16. The intraocular device of claim 1, wherein the mask has a curvature.

17. The intraocular device of claim 16, wherein the curvature of the mask is generally matched to the curvature of the intraocular lens.

18. The intraocular device of claim 1, wherein the mask comprises at least one haptic configured to support the mask within the eye of a patient.

19. The intraocular device of claim 1, wherein the aperture of the mask comprises a diameter of about 1.2 mm to about 2.0 mm.

20. The intraocular device of claim 1, wherein the mask comprises an outer diameter between about 3.2 mm and 3.8 mm.

21. The intraocular device of claim 1, further comprising the intraocular lens, the intraocular lens being connected to the mask by the one or more connectors.

22. The intraocular device of claim 1, wherein the aperture comprises a generally circular shape.

23. The intraocular device of claim 1, wherein the aperture comprises a generally oval shape.

24. A method of implanting an intraocular device, the method comprising:
creating a surgical incision in an eye;
implanting an intraocular lens in an intraocular space;
implanting an intraocular device adjacent to the intraocular lens, the intraocular device comprising a mask configured to increase the depth of focus of a patient, the mask comprising an aperture configured to transmit substantially all visible light and a non-transmissive region surrounding the aperture, the non-transmissive region configured to be substantially opaque to visible light.

25-45. (canceled)
